(19) United States
(12) Patent Application Publication
(10) Pub. No.: US 2023/0094303 A1
(43) Pub. Date: Mar. 30, 2023

(54) Methods and Systems Involving Digestible Primers for Improving Single Cell Multi-Omic Analysis

(71) Applicant: Mission Bio, Inc., South San Francisco, CA (US)

(72) Inventors: Dalia Dhingra, South San Francisco, CA (US); David Ruff, South San Francisco, CA (US)

(21) Appl. No.: 17/799,495

(22) PCT Filed: Feb. 12, 2021
(86) PCT No.: PCT/US21/17822

§ 371 Date: Aug. 12, 2022

Related U.S. Application Data
(60) Provisional application No. 62/975,361, filed on Feb. 12, 2020.

(51) Int. Cl. C12N 15/10; C12P 19/34

(52) U.S. Cl. CPC C12N 15/1096; C12P 19/34; C12Y 301/26004

(57) ABSTRACT

Digestible primers are incorporated into single cell analysis workflows to reduce and/or eliminate primer byproducts and misprimed nucleic acids. Specifically, digestible primers can participate in a first reaction, such as reverse transcription of RNA transcripts to generate cDNA, but digestible primers are digested to prevent them from participating in subsequent reactions, such as nucleic acid amplification. For example, digestible primers can include a primer with one or more ribonucleotide nucleobases, a primer with uracil bases, a primer with deoxyuridine sequences, or a primer with ribouridine sequences. Such primers can then be digested (e.g., enzymatically digested) to remove them from interfering in subsequent nucleic acid amplification reactions.

### CROSS REFERENCE

This application claims the benefit of and priority to U.S. Provisional Patent Application No. 62/975,361 filed Feb. 12, 2020, the entire disclosure of which is hereby incorporated by reference in its entirety for all purposes.

### BACKGROUND

A challenge in high throughput single-cell RNA sequencing where reverse transcription is followed by amplification is the generation of primer byproducts and mispriming of DNA by the reverse transcription primers. These primer byproducts and misprimed nucleic acids can be problematic as they can result in erroneous sequence reads and/or inaccurate characterization of individual cells. In other words, in scenarios such as multi-omic (e.g., RNA and DNA) single cell analysis, the presence of primer byproducts and/or misprimed nucleic acids results in qualitatively poor analysis of single cells.

### SUMMARY

The disclosure generally relates to methods and apparati for single-cell analysis through the implementation of digestible primers. In various embodiments, the digestible primers participate in a first reaction, such as a reverse transcription reaction involving RNA transcripts, and are subsequently digested. Therefore, the digestible primers cannot participate in a second reaction, such as a nucleic acid amplification reaction. Altogether, the implementation of digestible primers and their subsequent digestion represents an improved single-cell analysis workflow which, in particular embodiments involves a multi-omic single-cell analysis workflow (e.g., DNA and RNA analysis), which achieves improved sequence read metrics (e.g., improved percentage of reads after trimming, improved percentage of mapped reads, and/or improved percentage of reads with a valid cell barcode).

Disclosed herein is a method for generating a nucleic acid library, the method comprising: obtaining RNA and DNA from a single cell within a droplet; priming the RNA from the single cell using a digestible primer within the droplet; generating cDNA comprising the digestible primer from the primed RNA within the droplet; digesting the digestible primer; and sequencing at least the cDNA and the DNA of the single cell or sequences derived from the cDNA and the DNA of the single cell.

In various embodiments, the digestible primer comprises one of: A) one or more ribonucleotide nucleobases, B) one or more uracil nucleobases, C) a repeating deoxyuridine sequence, or D) a repeating ribouridine sequence, wherein digesting the digestible primer occurs subsequent to generating the cDNA and prior to a second cycle of nucleic acid amplification, wherein digesting the digestible primer comprises exposing the digestible primer to a RNase or uracil-DNA glycosylase.

In various embodiments, the digestible primer comprises one or more ribonucleotide nucleobases. In various embodiments, the digestible primer comprises a combination of ribonucleotides and deoxyribonucleotides. In various embodiments, the digestible primer comprises a ribonucleotide nucleobase every 2 nucleobases. In various embodiments, the digestible primer comprises a ribonucleotide nucleobase every 3 nucleobases. In various embodiments, the digestible primer comprises a ribonucleotide nucleobase every 4 nucleobases. In various embodiments, the digestible primer comprises a ribonucleotide nucleobase every 5 nucleobases, every 6 nucleobases, every 7 nucleobases, every 8 nucleobases, every 9 nucleobases, or every 10 nucleobases.

In various embodiments, the digestible primer comprises at least 3 consecutive ribouridine nucleobases. In various embodiments, the digestible primer comprises between 5 and 30 consecutive ribouridine nucleobases. In various embodiments, digesting the digestible primer comprises exposing the digestible primer to a RNase. In various embodiments, the RNase is one of RNase A or RNase H.

In various embodiments, the digestible primer comprises one or more uracil nucleobases. In various embodiments, the digestible primer comprises a uracil nucleobase every 3 nucleobases. In various embodiments, the digestible primer comprises a uracil nucleobase every 4 nucleobases. In various embodiments, the digestible primer comprises a uracil nucleobase every 5 nucleobases, every 6 nucleobases, every 7 nucleobases, every 8 nucleobases, every 9 nucleobases, or every 10 nucleobases. In various embodiments, the digestible primer comprises at least 3 consecutive deoxyuridine nucleobases. In various embodiments, the digestible primer comprises between 5 and 30 consecutive deoxyuridine nucleobases. In various embodiments, digesting the digestible primer comprises exposing the digestible primer to uracil-DNA glycosylase.

In various embodiments, generating cDNA comprising the digestible primer from the primed RNA comprises reverse transcribing the primed RNA. In various embodiments, digesting the digestible primer occurs within a second droplet. In various embodiments, digesting the digestible primer occurs subsequent to a first cycle of nucleic acid amplification.

In various embodiments, subsequent to generating cDNA and prior to digesting the digestible primer: the method comprises synthesizing a nucleic acid product derived from the cDNA, the nucleic acid product further comprising a sequence derived from a sequence of the digestible primer.

In various embodiments, digesting the digestible primer occurs prior to a first cycle of nucleic acid amplification. In various embodiments, subsequent to digesting the digestible primer: synthesizing a nucleic acid product derived from the cDNA, the nucleic acid product lacking a sequence derived from a sequence of the digestible primer; and priming the synthesized nucleic acid using a second primer different from the digestible primer. In various embodiments, the second primer is a gene specific primer. In various embodiments, the sequencing is a targeted sequencing.

In various embodiments, prior to digesting the digestible primer: the method comprises priming the cDNA using a random primer; and synthesizing a nucleic acid product derived from the cDNA, the nucleic acid product further comprising a sequence derived from a sequence of the digestible primer. In various embodiments, digesting the digestible primer occurs within a droplet. In various embodiments, digesting the digestible primer occurs within a second droplet. In various embodiments, the sequencing is a whole transcriptome sequencing.

In various embodiments, methods disclosed herein further comprise: subsequent to digesting the digestible primer, performing nucleic acid amplification to generate cDNA and gDNA amplicons. In various embodiments, performing nucleic acid amplification comprises incorporating cellular barcodes that indicate the single cell of origin, thereby generating cDNA amplicons comprising the cellular barcodes.

In various embodiments, obtaining RNA from a single cell within a droplet comprises: encapsulating the single cell in the droplet comprising reagents; lysing the single cell within the droplet; and exposing the lysed cell to conditions sufficient to release DNA from packaged chromatin. In various embodiments, the reagents comprise proteinase K, and wherein exposing the lysed cell comprising exposing the lysed cell to proteinase K to release DNA from packaged chromatin. In various embodiments, sequencing at least the cDNA of the single cell results in at least a 2-fold, at least a 3-fold, at least a 4-fold, or at least a 5-fold increase in percentage of mapped reads in comparison to a workflow process that implements oligo dT primers as opposed to digestible primers. In various embodiments, sequencing at least the cDNA of the single cell results in at least a 2-fold, at least a 3-fold, at least a 4-fold, or at least a 5-fold increase in percentage of reads with a valid barcode in comparison to a workflow process that implements oligo dT primers as opposed to digestible primers.

Additionally disclosed herein is a system for generating a nucleic acid library, the system comprising: a device configured to perform steps comprising: obtaining RNA and DNA from a single cell within a droplet; priming the RNA from the single cell using a digestible primer within the droplet; generating cDNA comprising the digestible primer from the primed RNA within the droplet; digesting the digestible primer; and sequencing at least the cDNA and the DNA of the single cell or sequences derived from the cDNA and the DNA of the single cell.

In various embodiments, the digestible primer comprises one of: A) one or more ribonucleotide nucleobases, B) one or more uracil nucleobases, C) a repeating deoxyuridine sequence, or D) a repeating ribouridine sequence, wherein digesting the digestible primer occurs subsequent to generating the cDNA and prior to a second cycle of nucleic acid amplification, wherein digesting the digestible primer comprises exposing the digestible primer to a RNase or uracil-DNA glycosylase.

In various embodiments, the digestible primer comprises one or more ribonucleotide nucleobases. In various embodiments, the digestible primer comprises a combination of ribonucleotides and deoxyribonucleotides. In various embodiments, the digestible primer comprises a ribonucleotide nucleobase every 2 nucleobases. In various embodiments, the digestible primer comprises a ribonucleotide nucleobase every 3 nucleobases. In various embodiments, the digestible primer comprises a ribonucleotide nucleobase every 4 nucleobases. In various embodiments, the digestible primer comprises a ribonucleotide nucleobase every 5 nucleobases, every 6 nucleobases, every 7 nucleobases, every 8 nucleobases, every 9 nucleobases, or every 10 nucleobases. In various embodiments, the digestible primer comprises at least 3 consecutive ribouridine nucleobases. In various embodiments, the digestible primer comprises between 5 and 30 consecutive ribouridine nucleobases. In various embodiments, digesting the digestible primer comprises exposing the digestible primer to a RNase. In various embodiments, the RNase is one of RNase A or RNase H.

In various embodiments, the digestible primer comprises one or more uracil nucleobases. In various embodiments, the digestible primer comprises a uracil nucleobase every 3 nucleobases. In various embodiments, the digestible primer comprises a uracil nucleobase every 4 nucleobases. In various embodiments, the digestible primer comprises a uracil nucleobase every 5 nucleobases, every 6 nucleobases, every 7 nucleobases, every 8 nucleobases, every 9 nucleobases, or every 10 nucleobases. In various embodiments, the digestible primer comprises at least 3 consecutive deoxyuridine nucleobases. In various embodiments, the digestible primer comprises between 5 and 30 consecutive deoxyuridine nucleobases. In various embodiments, digesting the digestible primer comprises exposing the digestible primer to uracil-DNA glycosylase.

In various embodiments, generating cDNA comprising the digestible primer from the primed RNA comprises reverse transcribing the primed RNA. In various embodiments, digesting the digestible primer occurs within a second droplet. In various embodiments, digesting the digestible primer occurs subsequent to a first cycle of nucleic acid amplification.

In various embodiments, subsequent to generating cDNA and prior to digesting the digestible primer, the device is configured to perform steps comprising: synthesizing a nucleic acid product derived from the cDNA, the nucleic acid product further comprising a sequence derived from a sequence of the digestible primer. In various embodiments, digesting the digestible primer occurs prior to a first cycle of nucleic acid amplification. In various embodiments, subsequent to digesting the digestible primer, the device is configured to perform steps comprising: synthesizing a nucleic acid product derived from the cDNA, the nucleic acid product lacking a sequence derived from a sequence of the digestible primer; and priming the synthesized nucleic acid using a second primer different from the digestible primer. In various embodiments, the second primer is a gene specific primer. In various embodiments, the sequencing is a targeted sequencing.

In various embodiments, prior to digesting the digestible primer: priming the cDNA using a random primer; and synthesizing a nucleic acid product derived from the cDNA, the nucleic acid product further comprising a sequence derived from a sequence of the digestible primer. In various embodiments, digesting the digestible primer occurs within a droplet. In various embodiments, digesting the digestible primer occurs within a second droplet. In various embodiments, the sequencing is a whole genome sequencing.

In various embodiments, the device is further configured to perform steps comprising: subsequent to digesting the digestible primer, performing nucleic acid amplification on the cDNA to generate cDNA amplicons. In various embodiments, performing nucleic acid amplification comprises incorporating cellular barcodes that indicate the single cell of origin, thereby generating cDNA amplicons comprising the cellular barcodes.

In various embodiments, obtaining RNA from a single cell within a droplet comprises: encapsulating the single cell in the droplet comprising reagents; lysing the single cell within the droplet; and exposing the lysed cell to conditions sufficient to release DNA from packaged chromatin. In various embodiments, the reagents comprise proteinase K, and wherein exposing the lysed cell comprising exposing the lysed cell to proteinase K to release DNA from packaged chromatin. In various embodiments, sequencing at least the cDNA of the single cell results in at least a 2-fold, at least a 3-fold, at least a 4-fold, or at least a 5-fold increase in percentage of mapped reads in comparison to a workflow process that implements oligo dT primers as opposed to digestible primers. In various embodiments, sequencing at least the cDNA of the single cell results in at least a 2-fold, at least a 3-fold, at least a 4-fold, or at least a 5-fold increase in percentage of reads with a valid barcode in comparison to a workflow process that implements oligo dT primers as opposed to digestible primers.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A shows an overall system environment for analyzing cell(s) through a single cell workflow analysis, in accordance with an embodiment.

FIG. 1B depicts a single cell workflow analysis to generate amplified nucleic acid molecules for sequencing, in accordance with an embodiment.

FIG. 2 is a flow process for analyzing nucleic acid sequences derived from analytes of the single cell, in accordance with an embodiment.

FIGS. 3A-3C depict the processing and releasing of analytes of a single cell in a droplet, in accordance with an embodiment.

FIG. 4A depicts the processing of RNA and gDNA in a first droplet, in accordance with an embodiment for targeted transcriptome sequencing.

FIG. 4B depicts the amplification and barcoding of nucleic acids derived from RNA and gDNA, in accordance with the embodiment shown in FIG. 4A.

FIG. 5A depicts the processing of RNA and gDNA in a first droplet, in accordance with an embodiment for nested targeted transcriptome sequencing.

FIG. 5B depicts the amplification and barcoding of nucleic acids derived from RNA and gDNA, in accordance with the embodiment shown in FIG. 5A.

FIG. 6A depicts the processing of RNA and gDNA in a first droplet, in accordance with a first embodiment for whole transcriptome sequencing.

FIG. 6B depicts the amplification and barcoding of nucleic acids derived from RNA and gDNA, in accordance with the embodiment shown in FIG. 6A.

FIG. 7A depicts the processing of RNA and gDNA in a first droplet, in accordance with a second embodiment for whole transcriptome sequencing

FIG. 7B depicts the amplification and barcoding of nucleic acids derived from RNA and gDNA, in accordance with the embodiment shown in FIG. 7A.

FIG. 8 depicts an example computing device for implementing system and methods described in reference to FIGS. 1-7.

FIG. 9A depicts generated products as a result of implementation of DNA base primers for targeted RNA sequencing.

FIG. 9B depicts generated products as a result of implementation of ribonucleotide primers for targeted RNA sequencing.

FIG. 9C depicts quantitative amounts of generated products as a result of implementation of deoxyribonucleotide or ribonucleotide primers for targeted sequencing.

FIG. 10A depicts qPCR and melting temperature plots identifying generated products as a result of implementation of uracil primers for whole transcriptome sequencing.

FIG. 10B depicts generated products as a result of implementing various concentrations of uracil-DNA glycosylase (UDG) enzyme.

FIGS. 11A-11C depict generated products as a result of implementing oligo dT, oligo dU, or oligo rU primers.

FIG. 11D depicts qPCR and melting temperature plots identifying generated products as a result of implementing oligo dT, oligo dU, or oligo rU primers for whole transcriptome sequencing.

### DETAILED DESCRIPTION

### Definitions

Terms used in the claims and specification are defined as set forth below unless otherwise specified.

The term “subject” or “patient” are used interchangeably and encompass an organism, human or non-human, mammal or non-mammal, male or female.

The term “sample” or “test sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art.

The term “analyte” refers to a component of a cell. Cell analytes can be informative for characterizing a cell. Therefore, performing single-cell analysis of one or more analytes of a cell using the systems and methods described herein are informative for determining a state or behavior of a cell. Examples of an analyte include a nucleic acid (e.g., RNA, DNA, cDNA), a protein, a peptide, an antibody, an antibody fragment, a polysaccharide, a sugar, a lipid, a small molecule, or combinations thereof. In particular embodiments, a single-cell analysis involves analyzing two different analytes such as RNA and DNA. In particular embodiments, a single-cell analysis involves analyzing three or more different analytes of a cell, such as RNA, DNA, and protein.

In some embodiments, the discrete entities as described herein are droplets. The terms “emulsion,” “drop,” “droplet,” and “microdroplet” are used interchangeably herein, to refer to small, generally spherically structures, containing at least a first fluid phase, e.g., an aqueous phase (e.g., water), bounded by a second fluid phase (e.g., oil) which is immiscible with the first fluid phase. In some embodiments, droplets according to the present disclosure may contain a first fluid phase, e.g., oil, bounded by a second immiscible fluid phase, e.g. an aqueous phase fluid (e.g., water). In some embodiments, the second fluid phase will be an immiscible phase carrier fluid. Thus droplets according to the present disclosure may be provided as aqueous-in-oil emulsions or oil-in-aqueous emulsions. Droplets may be sized and/or shaped as described herein for discrete entities. For example, droplets according to the present disclosure generally range from 1 μm to 1000 μm, inclusive, in diameter. Droplets according to the present disclosure may be used to encapsulate cells, nucleic acids (e.g., DNA), enzymes, reagents, reaction mixture, and a variety of other components. The term emulsion may be used to refer to an emulsion produced in, on, or by a microfluidic device and/or flowed from or applied by a microfluidic device.

“Complementarity” or “complementary” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See, e.g., Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

The terms “amplify,” “amplifying,” “amplification reaction” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further include any of the amplification processes known to one of ordinary skill in the art. In some embodiments, the amplification reaction includes polymerase chain reaction (PCR). In some embodiments, the amplification reaction includes an isothermal amplification reaction such as LAMP. In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification. The polynucleic acid produced by the amplification technology employed is generically referred to as an “amplicon” or “amplification product.”

Any nucleic acid amplification method may be utilized, such as a PCR-based assay, e.g., quantitative PCR (qPCR), or an isothermal amplification may be used to detect the presence of certain nucleic acids, e.g., genes of interest, present in discrete entities or one or more components thereof, e.g., cells encapsulated therein. Such assays can be applied to discrete entities within a microfluidic device or a portion thereof or any other suitable location. The conditions of such amplification or PCR-based assays may include detecting nucleic acid amplification over time and may vary in one or more ways.

A number of nucleic acid polymerases can be used in the amplification reactions utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

“Forward primer binding site” and “reverse primer binding site” refer to the regions on the template nucleic acid and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers.”

A “barcode” nucleic acid identification sequence can be incorporated into a nucleic acid primer or linked to a primer to enable independent sequencing and identification to be associated with one another via a barcode which relates information and identification that originated from molecules that existed within the same sample. There are numerous techniques that can be used to attach barcodes to the nucleic acids within a discrete entity. For example, the target nucleic acids may or may not be first amplified and fragmented into shorter pieces. The molecules can be combined with discrete entities, e.g., droplets, containing the barcodes. The barcodes can then be attached to the molecules using, for example, splicing by overlap extension. In this approach, the initial target molecules can have “adaptor” or “constant” sequences added, which are molecules of a known sequence to which primers can be synthesized. When combined with the barcodes, primers can be used that are complementary to the adaptor sequences and the barcode sequences, such that the product amplicons of both target nucleic acids and barcodes can anneal to one another and, via an extension reaction such as DNA polymerization, be extended onto one another, generating a double-stranded product including the target nucleic acids attached to the barcode sequence. Alternatively, the primers that amplify that target can themselves be barcoded so that, upon annealing and extending onto the target, the amplicon produced has the barcode sequence incorporated into it. This can be applied with a number of amplification strategies, including specific amplification with PCR or non-specific amplification with, for example, MDA. An alternative enzymatic reaction that can be used to attach barcodes to nucleic acids is ligation, including blunt or sticky end ligation. In this approach, the DNA barcodes are incubated with the nucleic acid targets and ligase enzyme, resulting in the ligation of the barcode to the targets. The ends of the nucleic acids can be modified as needed for ligation by a number of techniques, including by using adaptors introduced with ligase or fragments to enable greater control over the number of barcodes added to the end of the molecule.

The terms “identity” and “identical” and their variants, as used herein, when used in reference to two or more sequences, refer to the degree to which the two or more sequences (e.g., nucleotide or polypeptide sequences) are the same. In the context of two or more sequences, the percent identity or homology of the sequences or subsequences thereof indicates the percentage of all monomeric units (e.g., nucleotides or amino acids) that are the same at a given position or region of the sequence (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The terms “nucleic acid,” “polynucleotides,” and “oligonucleotides” refer to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U’ denotes uridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient. A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample.

Primers and oligonucleotides used in embodiments herein comprise nucleotides. In some embodiments, a nucleotide may comprise any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some, or all of such moieties. For example, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281.

In some embodiments, the nucleotide comprises a label and referred to herein as a “labeled nucleotide”; the label of the labeled nucleotide is referred to herein as a “nucleotide label.” In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

The phrase “digestible primers” used herein refers to primers that participate in a first reaction, but can be digested to prevent them from participating in a second reaction. For example, digestible primers can be primers that participate in the reverse transcription of RNA transcripts to generate cDNA, but are later digested such that the digestible primers do not participate in subsequent reactions involving the cDNA (e.g., amplification of cDNA). In some embodiments, digestible primers are reverse primers. In some embodiments, digestible primers are gene specific primers. In particular embodiments, digestible primers have one of the following characteristics: A) one or more ribonucleotide nucleobases, B) one or more uracil nucleobases, C) a repeating deoxyuridine sequence (e.g., oligo dUracil or oligo dU), or D) a repeating ribo uridine sequence (e.g., oligo rUracil or oligo rU).

Overview

Described herein are embodiments for an improved single-cell analysis workflow that reduces and/or eliminates the presence of primer byproducts and misprimed nucleic acids. Generally, undesired primer byproducts or misprimed nucleic acids are problematic as they result in erroneous sequence reads and/or inaccurate characterization of individual cells. In various embodiments, primer byproducts and misprimed nucleic acids are reduced by implementing digestible primers and eliminating the digestible primers prior to nucleic acid amplification such that primer products and misprimed nucleic acids are removed from the subsequent sequencing analysis. In particular embodiments, the digestible primers participate in the reverse transcription of RNA transcripts, and are subsequently digested such that the digestible primers are not involved in the nucleic acid amplification. Altogether, the implementation of digestible primers followed by digestion of the digestible primers enables improved sequence read metrics (e.g., improved percentage of reads after trimming, improved percentage of mapped reads, and/or improved percentage of reads with a valid cell barcode).

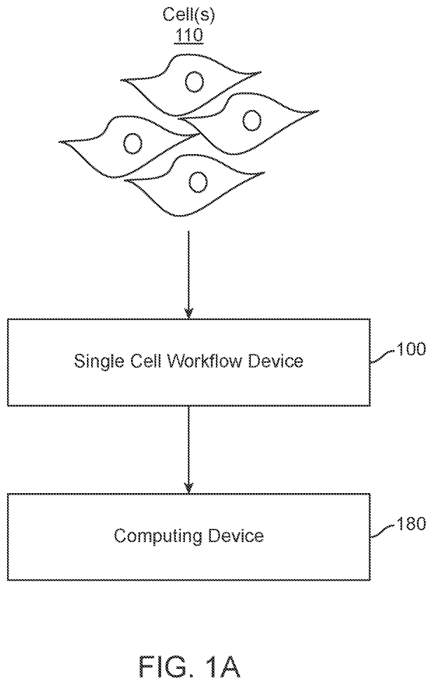

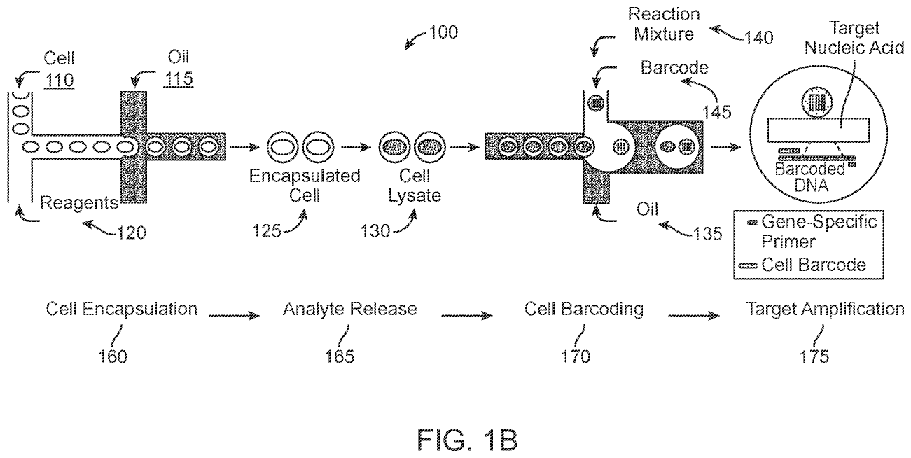

FIG. 1A shows an overall system environment for analyzing cell(s) through a single cell workflow analysis, in accordance with an embodiment. Generally, the single cell workflow device 100 is configured to process the cell(s) 110 and generate sequence reads derived from individual cell(s) 110. Further details as to the processes of the single cell workflow device 100 are described below in reference to FIG. 1B. The computing device 180 can analyze the sequence reads e.g., for purposes of building RNA/DNA libraries and/or characterizing individual cells. In various embodiments, the single cell workflow device 100 includes at least a microfluidic device that is configured to encapsulate cells with reagents to generate cell lysates comprising RNA and/or gDNA, encapsulate cell lysates with reaction mixtures, and perform nucleic acid amplification reactions. For example, the microfluidic device can include one or more fluidic channels that are fluidically connected. Therefore, the combining of an aqueous fluid through a first channel and a carrier fluid through a second channel results in the generation of emulsion droplets. In various embodiments, the fluidic channels of the microfluidic device may have at least one cross-sectional dimension on the order of a millimeter or smaller (e.g., less than or equal to about 1 millimeter). Additional details of microchannel design and dimensions is described in International Patent Application No. PCT/US2016/016444 and U.S. patent application Ser. No. 14/420,646, each of which is hereby incorporated by reference in its entirety. An example of a microfluidic device is the Tapestri™ Platform.

In various embodiments, the single cell workflow device 100 may also include one or more of: (a) a temperature control module for controlling the temperature of one or more portions of the subject devices and/or droplets therein and which is operably connected to the microfluidic device(s), (b) a detection means, i.e., a detector, e.g., an optical imager, operably connected to the microfluidic device(s), (c) an incubator, e.g., a cell incubator, operably connected to the microfluidic device(s), and (d) a sequencer operably connected to the microfluidic device(s). The one or more temperature and/or pressure control modules provide control over the temperature and/or pressure of a carrier fluid in one or more flow channels of a device. As an example, a temperature control module may be one or more thermal cycler that regulates the temperature for performing nucleic acid amplification. The one or more detection means i.e., a detector, e.g., an optical imager, are configured for detecting the presence of one or more droplets, or one or more characteristics thereof, including their composition. In some embodiments, detection means are configured to recognize one or more components of one or more droplets, in one or more flow channel. The sequencer is a hardware device configured to perform sequencing, such as next generation sequencing. Examples of sequencers include Illumina sequencers (e.g., MiniSeg™, MiSeg™, NextSeg™ 550 Series, or NextSeg™ 2000), Roche sequencing system 454, and Thermo Fisher Scientific sequencers (e.g., Ion GeneStudio S5 system, Ion Torrent Genexus System).

Reference is now made to FIG. 1B, which depicts an embodiment of processing single cells to generate amplified nucleic acid molecules for sequencing. Here, the processing of single cells can be performed by a single cell workflow device (e.g., the single cell workflow device 100 disclosed in FIG. 1A). Specifically, FIG. 1B depicts a workflow process including the steps of cell encapsulation 160, analyte release 165, cell barcoding 170, and target amplification 175 of target nucleic acid molecules.

Generally, the cell encapsulation step 160 involves encapsulating a single cell 110 with reagents 120 into a droplet. In various embodiments, the droplet is formed by partitioning aqueous fluid containing the cell 110 and reagents 120 into a carrier fluid (e.g., oil 115), thereby resulting in a aqueous fluid-in-oil emulsion. The droplet includes encapsulated cell 125 and the reagents 120. The encapsulated cell undergoes an analyte release at step 165. Generally, the reagents cause the cell to lyse, thereby generating a cell lysate 130 within the droplet. The cell lysate 130 includes the contents of the cell, which can include one or more different types of analytes (e.g., RNA transcripts, DNA, protein, lipids, or carbohydrates). In various embodiments, the different analytes of the cell lysate 130 can interact with reagents 120 within the droplet. For example, in particular embodiments, reverse transcriptase in the reagents 120 can reverse transcribe cDNA molecules from RNA transcripts that are present in the cell lysate 130.

In particular embodiments, the reagents 120 include primers. In some embodiments, the primers are gene specific primers. In various embodiments, the primers are reverse primers that are capable of hybridizing to a portion of a nucleic acid, such as a RNA transcript. In such embodiments, the primers enables the reverse transcription of RNA transcripts to generate cDNA. In particular embodiments, the primers are digestible primers. For example, digestible primers can participate in the reverse transcription of RNA transcripts to generate cDNA, but are later digested such that the digestible primers do not participate in subsequent reactions involving the cDNA (e.g., amplification of cDNA). Further details on digestible primers is described below. In some embodiments, the digestible primers are digested here in this droplet at step 165. In other embodiments, the digestible primers remain intact and are not digested here in the droplet at step 165.

The cell barcoding step 170 involves encapsulating the cell lysate 130 into a second droplet along with a barcode 145 and/or reaction mixture 140. In various embodiments, the second emulsion is formed by partitioning aqueous fluid containing the cell lysate 130 into immiscible oil 135. As shown in FIG. 1B, the reaction mixture 140 and barcode 145 can be introduced through a separate stream of aqueous fluid, thereby partitioning the reaction mixture 140 and barcode 145 into the second droplet along with the cell lysate 130.

Generally, the reaction mixture 140 enables the performance of a reaction, such as a nucleic acid amplification reaction. In various embodiments, the reaction mixture 140 includes one or more enzymes capable of digesting primers such that the nucleic acid amplification reaction can proceed with improved efficiency. In such embodiments where the reaction mixture 140 includes one or more enzymes capable of digesting the digestible primers, the enzymes digest the digestible primers here in this droplet at step 170. In other embodiments, the digestible primers are previously digested in the droplet at step 165 and therefore, need not be digested here at step 170. In various embodiments, the enzymes digest the digestible primers prior to a first cycle of nucleic acid amplification. In various embodiments, the enzymes digest the digestible primers subsequent to a first cycle of nucleic acid amplification. In various embodiments, the enzymes digest the digestible primers subsequent to a first cycle of nucleic acid amplification, but prior to a second cycle of nucleic acid amplification.

The target amplification step 175 involves amplifying target nucleic acids. For example, target nucleic acids of the cell lysate undergo amplification using the reaction mixture 140 in the second emulsion, thereby generating amplicons derived from the target nucleic acids. Generally, at step 175, any digestible primers that were previously introduced (e.g., previously introduced as part of the reagents 120) have been digested, thereby reducing or completely eliminating the presence of digestible primers. Therefore, digestible primers do not play a role in the target amplification 175 step.

Generally, a barcode 145 can label a target nucleic acid to be analyzed (e.g., an analyte of the cell lysate such as genomic DNA or cDNA that has been reverse transcribed from RNA), which enables subsequent identification of the origin of a sequence read that is derived from the target nucleic acid. In various embodiments, multiple barcodes 145 can label multiple target nucleic acid of the cell lysate, thereby enabling the subsequent identification of the origin of large quantities of sequence reads.

As referred herein, the workflow process shown in FIG. 1B is a two-step workflow process in which analyte release 165 from the cell occurs separate from the steps of cell barcoding 170 and target amplification 175. Specifically, analyte release 165 from a cell occurs within a first droplet followed by cell barcoding 170 and target amplification 175 in a second emulsion. In various embodiments, alternative workflow processes (e.g., workflow processes other than the two-step workflow process shown in FIG. 1A) can be employed. For example, the cell 110, reagents 120, reaction mixture 140, and barcode 145 can be encapsulated in a single emulsion. Thus, analyte release 165 can occur within the droplet, followed by cell barcoding 170 and target amplification 175 within the same droplet. Additionally, although FIG. 1B depicts cell barcoding 170 and target amplification 175 as two separate steps, in various embodiments, the target nucleic acid is labeled with a barcode 145 through the nucleic acid amplification step.

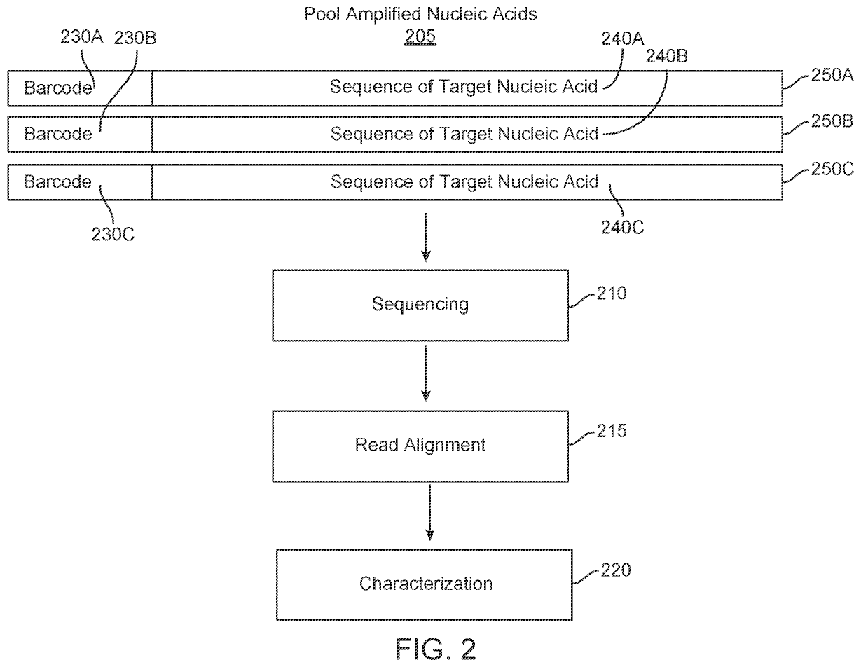

FIG. 2 is a flow process for analyzing nucleic acid sequences derived from analytes of the single cell, in accordance with an embodiment. Specifically, FIG. 2 depicts the steps of pooling amplified nucleic acids at step 205, sequencing the amplified nucleic acids at step 210, read alignment at step 215, and characterization at step 220. Generally, the flow process shown in FIG. 2 is a continuation of the workflow process shown in FIG. 1B.

For example, after target amplification at step 175 of FIG. 1B, the amplified nucleic acids 250A, 250B, and 250C are pooled at step 205 shown in FIG. 2. For example, individual droplets containing amplified nucleic acids are pooled and collected, and the immiscible oil of the emulsions is removed. Thus, amplified nucleic acids from multiple cells can be pooled together. FIG. 2 depicts three amplified nucleic acids 250A, 250B, and 250C. In various embodiments, pooled nucleic acids can include hundreds, thousands, or millions of nucleic acids derived from analytes of multiple cells.

In various embodiments, each amplified nucleic acid 250 includes at least a sequence of a target nucleic acid 240 and a barcode 230. In various embodiments, an amplified nucleic acid 250 can include additional sequences, such as any of a universal primer sequence, a random primer sequence, a gene specific primer forward sequence, a gene specific primer reverse sequence, a constant region, or sequencing adapters.

In various embodiments, the amplified nucleic acids 250A, 250B, and 250C are derived from the same single cell and therefore, the barcodes 230A, 230B, and 230C are the same. Therefore, sequencing of the barcodes 230 enables the determination that the amplified nucleic acids 250 are derived from the same cell. In various embodiments, the amplified nucleic acids 250A, 250B, and 250C are pooled and derived from different cells. Therefore, the barcodes 230A, 230B, and 230C are different from one another and sequencing of the barcodes 230 enables the determination that the amplified nucleic acids 250 are derived from different cells.

At step 210, the pooled amplified nucleic acids 250 undergo sequencing to generate sequence reads. For each of one or more amplicons, the sequence read includes at least the sequence of the barcode and the target nucleic acid. Sequence reads originating from individual cells are clustered according to the barcode sequences included in the amplicons. At step 215, the sequence reads for each single cell are aligned (e.g., to a reference genome). Aligning the sequence reads to the reference genome enables the determination of where in the genome the sequence read is derived from. For example, multiple sequence reads generated from amplicons derived from a RNA transcript molecule, when aligned to a position of the genome, can reveal that a gene at the position of the genome was transcribed. As another example, multiple sequence reads generated amplicons derived from a genomic DNA molecule, when aligned to a position of the genome, can reveal the sequence of the gene at the position of the genome.

The alignment of sequence reads at step 215 generates libraries, such as single cell DNA libraries or single cell RNA libraries. Therefore, at step 220, characterization of the libraries and/or the single cells can be performed. In various embodiments, characterization of a library (e.g., DNA library or RNA library) can involve determining sequencing metrics including, but not limited to: percentage of reads after trimming, percentage of primer reads (e.g., percentage of oligo dT/dU reads), percentage of reads with a particular forward primer, percentage of mapped reads, percentage of reads with a valid cell barcode, percentage of exon reads, percentage of intron reads, percentage of mitochondrial reads, and percentage or rRNA reads. In various embodiments, characterization of single cells can involve identifying one or more mutations (e.g., allelic variants, point mutations, single nucleotide variations/polymorphisms, translocations, DNA/RNA fusions, loss of heterozygosity) that are present in one or more of the single cells. Further description regarding characterization of single cells is described in PCT/US2020/026480 and PCT/US2020/026482, each of which is hereby incorporated by reference in its entirety.

Methods for Performing Single-Cell Analysis

Encapuslation, Analyte Release, Barcoding, and Amplification

Embodiments described herein involve encapsulating one or more cells (e.g., at step 160 in FIG. 1B) to perform single-cell analysis on the one or more cells. In various embodiments, the one or more cells can be isolated from a test sample obtained from a subject or a patient. In various embodiments, the one or more cells are healthy cells taken from a healthy subject. In various embodiments, the one or more cells include cancer cells taken from a subject previously diagnosed with cancer. For example, such cancer cells can be tumor cells available in the bloodstream of the subject diagnosed with cancer. Thus, single-cell analysis of the tumor cells enables cellular and sub-cellular prediction of the subject's cancer. In various embodiments, the test sample is obtained from a subject following treatment of the subject (e.g., following a therapy such as cancer therapy). Thus, single-cell analysis of the cells enables cellular and sub-cellular prediction of the subject's response to a therapy.

In various embodiments, encapsulating a cell with reagents is accomplished by combining an aqueous phase including the cell and reagents with an immiscible oil phase. In one embodiment, an aqueous phase including the cell and reagents are flowed together with a flowing immiscible oil phase such that water in oil emulsions are formed, where at least one emulsion includes a single cell and the reagents. In various embodiments the immiscible oil phase includes a fluorous oil, a fluorous non-ionic surfactant, or both. In various embodiments, emulsions can have an internal volume of about 0.001 to 1000 picoliters or more and can range from 0.1 to 1000 μm in diameter.

In various embodiments, the aqueous phase including the cell and reagents need not be simultaneously flowing with the immiscible oil phase. For example, the aqueous phase can be flowed to contact a stationary reservoir of the immiscible oil phase, thereby enabling the budding of water in oil emulsions within the stationary oil reservoir.

In various embodiments, combining the aqueous phase and the immiscible oil phase can be performed in a microfluidic device. For example, the aqueous phase can flow through a microchannel of the microfluidic device to contact the immiscible oil phase, which is simultaneously flowing through a separate microchannel or is held in a stationary reservoir of the microfluidic device. The encapsulated cell and reagents within an emulsion can then be flowed through the microfluidic device to undergo cell lysis.

Further example embodiments of adding reagents and cells to emulsions can include merging emulsions that separately contain the cells and reagents or picoinjecting reagents into an emulsion. Further description of example embodiments is described in U.S. application Ser. No. 14/420,646, which is hereby incorporated by reference in its entirety.

Generally, the encapsulated cell in an emulsion is lysed to generate cell lysate. In various embodiments, the cell is lysed due to the reagents which include one or more lysing agents that cause the cell to lyse. Examples of lysing agents include detergents such as Triton X-100, NP-40 (e.g., Tergitol-type NP-40 or nonyl phenoxypolyethoxylethanol), as well as cytotoxins. Examples of NP-40 include Thermo Scientific NP-40 Surfact-Amps Detergent solution and Sigma Aldrich NP-40 (TERGITOL Type NP-40). In some embodiments, cell lysis may also, or instead, rely on techniques that do not involve a lysing agent in the reagent. For example, lysis may be achieved by mechanical techniques that may employ various geometric features to effect piercing, shearing, abrading, etc. of cells. Other types of mechanical breakage such as acoustic techniques may also be used. Further, thermal energy can also be used to lyse cells. Any convenient means of effecting cell lysis may be employed in the methods described herein.

In various embodiments, the reagents include reverse transcriptase which reverse transcribes mRNA transcripts released from the cell to generate corresponding cDNA and further include primers that hybridize with mRNA transcripts, thereby enabling the reverse transcription reaction to occur. In various embodiments, such primers are digestible primers that participate in the reverse transcription reaction, but are subsequently digested to prevent their participation in subsequent reactions.

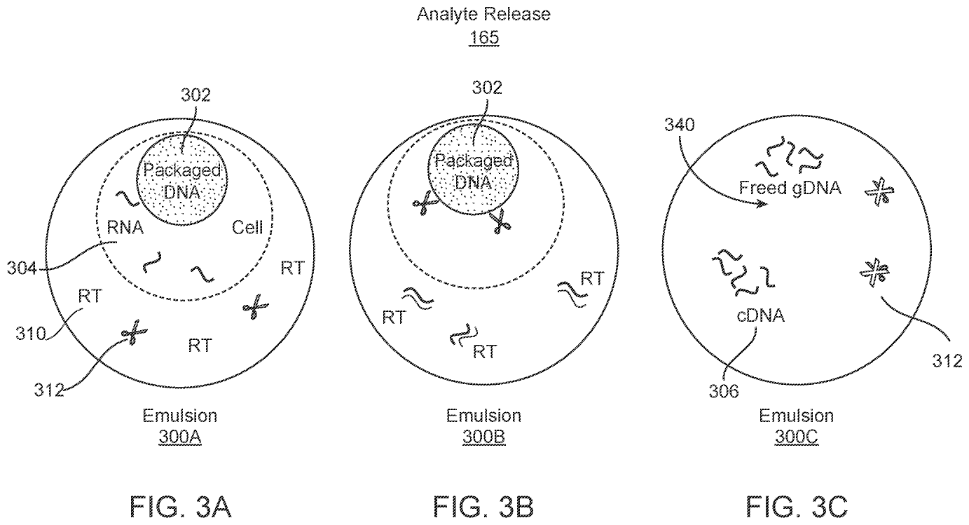

FIGS. 3A-3C depict the processing and releasing of analytes of a single cell in a droplet, in accordance with an embodiment. In FIG. 3A, the cell is lysed, as indicated by the dotted line of the cell membrane. In some embodiments, the reagents include a detergent, such as NP40 (e.g., 0.01% or 1.0% NP40) or Triton-X100, which causes the cell to lyse. The lysed cell includes analytes such as RNA transcripts within the cytoplasm of the cell as well as packaged DNA 302, which refers to the organization of DNA with histones, thereby forming nucleosomes that are packaged as chromatin. As shown in FIG. 3A, the reagents included in the emulsion 300A further includes reverse transcriptase (abbreviated as “RT” 310). Furthermore, the reagents included in the emulsion 300A further includes an enzyme 312 that digests the packaged DNA 302. In various embodiments, the enzyme 312 is proteinase K.

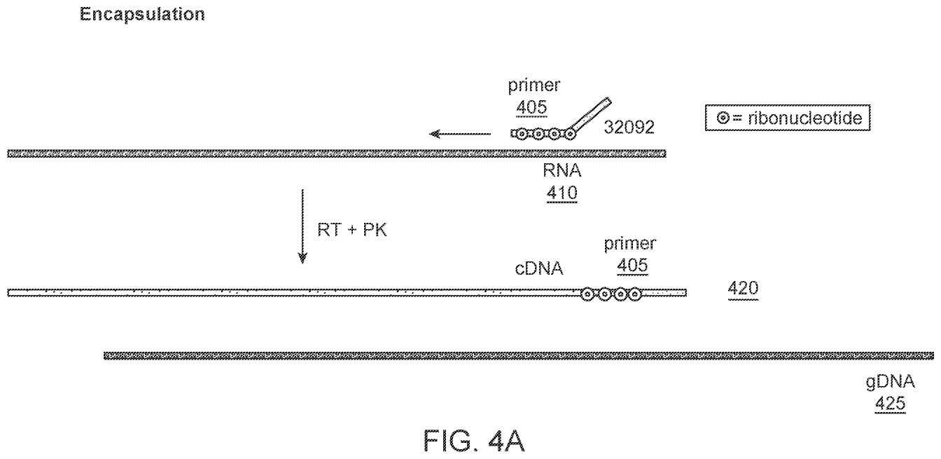

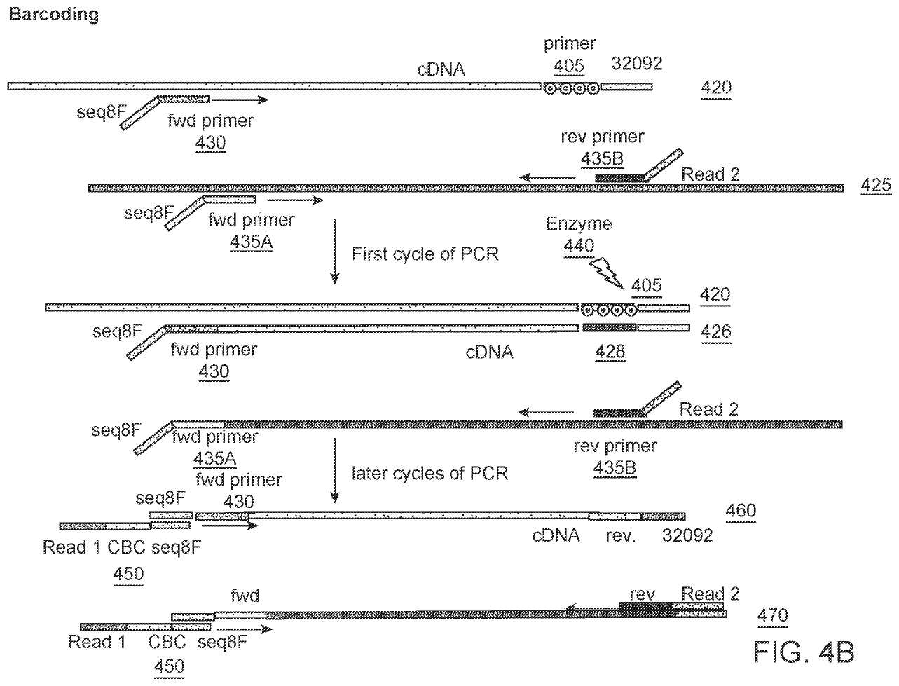

FIG. 3B depicts the emulsion 300B in a second state as reverse transcriptase performs reverse transcription on the RNA transcripts and the enzymes 312 digest the packaged DNA 302. In particular embodiments, reverse transcription occurs through the use of digestible primers. For example, a digestible primer can hybridize with a portion of RNA transcripts and reverse transcriptase generates a cDNA strand off of the RNA transcript. Example digestible primers have one of the following characteristics: A) one or more ribonucleotide nucleobases, B) one or more uracil nucleobases, C) a repeating deoxyuridine sequence (e.g., oligo dUracil or oligo dU), or D) a repeating ribouridine sequence (e.g., oligo rUracil or oligo rU). Various embodiments involving the implementation of digestible primers for generating cDNA nucleic acids are described in further detail below in reference to FIGS. 4A, 5A, 6A, and 7A.

FIG. 3C depicts the emulsion 300C in a third state that includes synthesized cDNA 306. FIG. 3C also depicts freed gDNA 340 that is released from the packaged DNA 302. In various embodiments, when transitioning between FIG. 3B and FIG. 3C, the digestible primers are digested. Namely, after the digestible primers have been used to prime and reverse transcribe the RNA 304, the digestible primers are digested to remove their participation in subsequent reactions. In various embodiments, the digestion of digestible primers reduces or eliminates presence of the digestible primers. This can include digestible primers that have formed primer byproducts and misprimed digestible primers (e.g., digestible primers that have primed a different nucleic acid such as the freed genomic DNA).

In various embodiments, the emulsion 300C can be exposed to conditions to inactivate the enzymes 312. In various embodiments, the emulsion 300C is exposed to an elevated temperature of at least 50° C. to inactivate the enzymes 312. In various embodiments, the emulsion 300C is exposed to an elevated temperature of at least 60° C. to inactivate the enzymes 312. In various embodiments, the emulsion 300C is exposed to an elevated temperature of at least 70° C. to inactivate the enzymes 312. In various embodiments, the emulsion 300C is exposed to an elevated temperature of at least 80° C. to inactivate the enzymes 312.

Returning to the step of cell barcoding 170 in FIG. 1B, it includes encapsulating a cell lysate 130 with a reaction mixture 140 and a barcode 145. Generally, the reaction mixture includes reactants sufficient for performing a reaction, such as nucleic acid amplification, on analytes of the cell lysate. In various embodiments, the reaction mixture 140 includes components, such as primers, for performing the nucleic acid reaction on the analytes. Such primers are capable of acting as a point of initiation of synthesis along a complementary strand when placed under conditions in which synthesis of a primer extension product which is complementary to a nucleic acid strand is catalyzed.

In various embodiments, a cell lysate is encapsulated with a reaction mixture and a barcode by combining an aqueous phase including the reaction mixture and the barcode with the cell lysate and an immiscible oil phase. In one embodiment, an aqueous phase including the reaction mixture and the barcode are flowed together with a flowing cell lysate and a flowing immiscible oil phase such that water in oil emulsions are formed, where at least one emulsion includes a cell lysate, the reaction mixture, and the barcode. In various embodiments the immiscible oil phase includes a fluorous oil, a fluorous non-ionic surfactant, or both. In various embodiments, emulsions can have an internal volume of about 0.001 to 1000 picoliters or more and can range from 0.1 to 1000 μm in diameter.

In various embodiments, combining the aqueous phase and the immiscible oil phase can be performed in a microfluidic device. For example, the aqueous phase can flow through a microchannel of the microfluidic device to contact the immiscible oil phase, which is simultaneously flowing through a separate microchannel or is held in a stationary reservoir of the microfluidic device. The encapsulated cell lysate, reaction mixture, and barcode within an emulsion can then be flowed through the microfluidic device to perform amplification of target nucleic acids.

Further example embodiments of adding reaction mixture and barcodes to emulsions can include merging emulsions that separately contain the cell lysate and reaction mixture and barcodes or picoinjecting the reaction mixture and/or barcode into an emulsion. Further description of example embodiments of merging emulsions or picoinjecting substances into an emulsion is found in U.S. application Ser. No. 14/420,646, which is hereby incorporated by reference in its entirety.

In various embodiments, subsequent to adding the reaction mixture and barcode to an emulsion, the digestible primers are digested. Digestible primers are digested to remove their subsequent participation in reactions such as nucleic acid amplification. In various embodiments, the digestion of digestible primers reduces or eliminates presence of the digestible primers. This can include digestible primers that have formed primer byproducts and misprimed digestible primers (e.g., digestible primers that have primed a different nucleic acid such as genomic DNA).

The emulsion may be incubated under conditions that facilitates the nucleic acid amplification reaction. In various embodiments, the emulsion may be incubated on the same microfluidic device as was used to add the reaction mixture and/or barcode, or may be incubated on a separate device. In certain embodiments, incubating the emulsion under conditions that facilitates nucleic acid amplification is performed on the same microfluidic device used to encapsulate the cells and lyse the cells. Incubating the emulsions may take a variety of forms. In certain aspects, the emulsions containing the reaction mix, barcode, and cell lysate may be flowed through a channel that incubates the emulsions under conditions effective for nucleic acid amplification. Flowing the microdroplets through a channel may involve a channel that snakes over various temperature zones maintained at temperatures effective for PCR. Such channels may, for example, cycle over two or more temperature zones, wherein at least one zone is maintained at about 65° C. and at least one zone is maintained at about 95° C. As the drops move through such zones, their temperature cycles, as needed for nucleic acid amplification. The number of zones, and the respective temperature of each zone, may be readily determined by those of skill in the art to achieve the desired nucleic acid amplification. Additionally, the extent of nucleic amplification can be controlled by modulating the concentration of the reactants in the reaction mixture. In some instances, this is useful for fine tuning of the reactions in which the amplified products are used.

In various embodiments, following nucleic acid amplification, emulsions containing the amplified nucleic acids are collected. In various embodiments, the emulsions are collected in a well, such as a well of a microfluidic device. In various embodiments, the emulsions are collected in a reservoir or a tube, such as an Eppendorf tube. Once collected, the amplified nucleic acids across the different emulsions are pooled. In one embodiment, the emulsions are broken by providing an external stimuli to pool the amplified nucleic acids. In one embodiment, the emulsions naturally aggregate over time given the density differences between the aqueous phase and immiscible oil phase. Thus, the amplified nucleic acids pool in the aqueous phase.

Following pooling, the amplified nucleic acids can undergo further preparation for sequencing. For example, sequencing adapters can be added to the pooled nucleic acids. Example sequencing adapters are P5 and P7 sequencing adapters. The sequencing adapters enable the subsequent sequencing of the nucleic acids.

Sequencing and Read Alignment

Amplified nucleic acids are sequenced to obtain sequence reads for generating a sequencing library. Sequence reads can be achieved with commercially available next generation sequencing (NGS) platforms, including platforms that perform any of sequencing by synthesis, sequencing by ligation, pyrosequencing, using reversible terminator chemistry, using phospholinked fluorescent nucleotides, or real-time sequencing. As an example, amplified nucleic acids may be sequenced on an Illumina MiSeq platform.

When pyrosequencing, libraries of NGS fragments are cloned in-situ amplified by capture of one matrix molecule using granules coated with oligonucleotides complementary to adapters. Each granule containing a matrix of the same type is placed in a microbubble of the “water in oil” type and the matrix is cloned amplified using a method called emulsion PCR. After amplification, the emulsion is destroyed and the granules are stacked in separate wells of a titration picoplate acting as a flow cell during sequencing reactions. The ordered multiple administration of each of the four dNTP reagents into the flow cell occurs in the presence of sequencing enzymes and a luminescent reporter, such as luciferase. In the case where a suitable dNTP is added to the 3′ end of the sequencing primer, the resulting ATP produces a flash of luminescence within the well, which is recorded using a CCD camera. It is possible to achieve a read length of more than or equal to 400 bases, and it is possible to obtain 10⁶ readings of the sequence, resulting in up to 500 million base pairs (megabytes) of the sequence. Additional details for pyrosequencing is described in Voelkerding et al., Clinical Chem., 55: 641-658, 2009; MacLean et al., Nature Rev. Microbiol., 7: 287-296; U.S. Pat. Nos. 6,210,891; 6,258,568; each of which is hereby incorporated by reference in its entirety.

On the Solexa/Illumina platform, sequencing data is produced in the form of short readings. In this method, fragments of a library of NGS fragments are captured on the surface of a flow cell that is coated with oligonucleotide anchor molecules. An anchor molecule is used as a PCR primer, but due to the length of the matrix and its proximity to other nearby anchor oligonucleotides, elongation by PCR leads to the formation of a “vault” of the molecule with its hybridization with the neighboring anchor oligonucleotide and the formation of a bridging structure on the surface of the flow cell. These DNA loops are denatured and cleaved. Straight chains are then sequenced using reversibly stained terminators. The nucleotides included in the sequence are determined by detecting fluorescence after inclusion, where each fluorescent and blocking agent is removed prior to the next dNTP addition cycle. Additional details for sequencing using the Illumina platform is found in Voelkerding et al., Clinical Chem., 55: 641-658, 2009; MacLean et al., Nature Rev. Microbiol., 7: 287-296; U.S. Pat. Nos. 6,833,246; 7,115,400; 6,969,488; each of which is hereby incorporated by reference in its entirety.

Sequencing of nucleic acid molecules using SOLiD technology includes clonal amplification of the library of NGS fragments using emulsion PCR. After that, the granules containing the matrix are immobilized on the derivatized surface of the glass flow cell and annealed with a primer complementary to the adapter oligonucleotide. However, instead of using the indicated primer for 3′extension, it is used to obtain a 5′ phosphate group for ligation for test probes containing two probe-specific bases followed by 6 degenerate bases and one of four fluorescent labels. In the SOLiD system, test probes have 16 possible combinations of two bases at the 3′end of each probe and one of four fluorescent dyes at the 5′ end. The color of the fluorescent dye and, thus, the identity of each probe, corresponds to a certain color space coding scheme. After many cycles of alignment of the probe, ligation of the probe and detection of a fluorescent signal, denaturation followed by a second sequencing cycle using a primer that is shifted by one base compared to the original primer. In this way, the sequence of the matrix can be reconstructed by calculation; matrix bases are checked twice, which leads to increased accuracy. Additional details for sequencing using SOLiD technology is found in Voelkerding et al., Clinical Chem., 55: 641-658, 2009; MacLean et al., Nature Rev. Microbiol., 7: 287-296; U.S. Pat. Nos. 5,912,148; 6,130,073; each of which is incorporated by reference in its entirety.

In particular embodiments, HeliScope from Helicos BioSciences is used. Sequencing is achieved by the addition of polymerase and serial additions of fluorescently-labeled dNTP reagents. Switching on leads to the appearance of a fluorescent signal corresponding to dNTP, and the specified signal is captured by the CCD camera before each dNTP addition cycle. The reading length of the sequence varies from 25-50 nucleotides with a total yield exceeding 1 billion nucleotide pairs per analytical work cycle. Additional details for performing sequencing using HeliScope is found in Voelkerding et al., Clinical Chem., 55: 641-658, 2009; MacLean et al., Nature Rev. Microbiol., 7: 287-296; U.S. Pat. Nos. 7,169,560; 7,282,337; 7,482,120; 7,501,245; 6,818,395; 6,911,345; 7,501,245; each of which is incorporated by reference in its entirety.

In some embodiments, a Roche sequencing system 454 is used. Sequencing 454 involves two steps. In the first step, DNA is cut into fragments of approximately 300-800 base pairs, and these fragments have blunt ends. Oligonucleotide adapters are then ligated to the ends of the fragments. The adapter serve as primers for amplification and sequencing of fragments. Fragments can be attached to DNA-capture beads, for example, streptavidin-coated beads, using, for example, an adapter that contains a 5′-biotin tag. Fragments attached to the granules are amplified by PCR within the droplets of an oil-water emulsion. The result is multiple copies of cloned amplified DNA fragments on each bead. At the second stage, the granules are captured in wells (several picoliters in volume). Pyrosequencing is carried out on each DNA fragment in parallel. Adding one or more nucleotides leads to the generation of a light signal, which is recorded on the CCD camera of the sequencing instrument. The signal intensity is proportional to the number of nucleotides included. Pyrosequencing uses pyrophosphate (PPi), which is released upon the addition of a nucleotide. PPi is converted to ATP using ATP sulfurylase in the presence of adenosine 5′phosphosulfate. Luciferase uses ATP to convert luciferin to oxyluciferin, and as a result of this reaction, light is generated that is detected and analyzed. Additional details for performing sequencing 454 is found in Margulies et al. (2005) Nature 437: 376-380, which is hereby incorporated by reference in its entirety.

Ion Torrent technology is a DNA sequencing method based on the detection of hydrogen ions that are released during DNA polymerization. The microwell contains a fragment of a library of NGS fragments to be sequenced. Under the microwell layer is the hypersensitive ion sensor ISFET. All layers are contained within a semiconductor CMOS chip, similar to the chip used in the electronics industry. When dNTP is incorporated into a growing complementary chain, a hydrogen ion is released that excites a hypersensitive ion sensor. If homopolymer repeats are present in the sequence of the template, multiple dNTP molecules will be included in one cycle. This results in a corresponding amount of hydrogen atoms being released and in proportion to a higher electrical signal. This technology is different from other sequencing technologies that do not use modified nucleotides or optical devices. Additional details for Ion Torrent Technology is found in Science 327 (5970): 1190 (2010); US Patent Application Publication Nos. 20090026082, 20090127589, 20100301398, 20100197507, 20100188073, and 20100137143, each of which is incorporated by reference in its entirety.

In various embodiments, sequencing reads obtained from the NGS methods can be filtered by quality and grouped by barcode sequence using any algorithms known in the art, e.g., Python script barcodeCleanup.py. In some embodiments, a given sequencing read may be discarded if more than about 20% of its bases have a quality score (Q-score) less than Q20, indicating a base call accuracy of about 99%. In some embodiments, a given sequencing read may be discarded if more than about 5%, about 10%, about 15%, about 20%, about 25%, about 30% have a Q-score less than Q10, Q20, Q30, Q40, Q50, Q60, or more, indicating a base call accuracy of about 90%, about 99%, about 99.9%, about 99.99%, about 99.999%, about 99.9999%, or more, respectively.

In some embodiments, all sequencing reads associated with a barcode containing less than 50 reads may be discarded to ensure that all barcode groups, representing single cells, contain a sufficient number of high-quality reads. In some embodiments, all sequencing reads associated with a barcode containing less than 30, less than 40, less than 50, less than 60, less than 70, less than 80, less than 90, less than 100 or more may be discarded to ensure the quality of the barcode groups representing single cells.

Sequence reads with common barcode sequences (e.g., meaning that sequence reads originated from the same cell) may be aligned to a reference genome using known methods in the art to determine alignment position information. The alignment position information may indicate a beginning position and an end position of a region in the reference genome that corresponds to a beginning nucleotide base and end nucleotide base of a given sequence read. A region in the reference genome may be associated with a target gene or a segment of a gene. Example aligner algorithms include BWA, Bowtie, Spliced Transcripts Alignment to a Reference (STAR), Tophat, or HISAT2. Further details for aligning sequence reads to reference sequences is described in U.S. application Ser. No. 16/279,315, which is hereby incorporated by reference in its entirety. In various embodiments, an output file having SAM (sequence alignment map) format or BAM (binary alignment map) format may be generated and output for subsequent analysis.

In various embodiments, sequencing and read alignment results in generation of a nucleic acid library (e.g., a RNA library and/or a DNA library). In various embodiments, nucleic acid libraries can be evaluated based on one or more sequence read metrics. Example sequence read metrics include percentage of reads after trimming, percentage of oligo dT/dU reads, percentage of reads with the forward primer, percentage of mapped reads, and percentage of reads with a valid cell barcode. Generally, the single cell analysis workflow disclosed herein involving the implementation of digestible primers followed by digestion of the digestible primers enables improved sequence read metrics in comparison to a single cell analysis workflow that does not implement digestible primers. An example single cell analysis workflow that does not implement digestible primers can be a workflow that implements an oligo dT primer that enables reverse transcription, and is not subsequently digested.

In various embodiments, the single cell analysis workflow disclosed herein involving the implementation of digestible primers followed by digestion of the digestible primers achieves at least a 2-fold increase in percentage of mapped reads in comparison to a workflow process that implements oligo dT primers as opposed to digestible primers. In various embodiments, the single cell analysis workflow disclosed herein involving the implementation of digestible primers followed by digestion of the digestible primers achieves at least a 3-fold increase, at least a 4-fold increase, or at least a 5-fold increase in percentage of mapped reads in comparison to a workflow process that implements oligo dT primers as opposed to digestible primers.

In various embodiments, the single cell analysis workflow disclosed herein involving the implementation of digestible primers followed by digestion of the digestible primers achieves at least a 1.2-fold increase in percentage of reads after trimming in comparison to a workflow process that implements oligo dT primers as opposed to digestible primers. In various embodiments, the single cell analysis workflow disclosed herein involving the implementation of digestible primers followed by digestion of the digestible primers achieves at least a 2-fold increase, at least a 3-fold increase, at least a 4-fold increase, or at least a 5-fold increase in percentage of reads after trimming in comparison to a workflow process that implements oligo dT primers as opposed to digestible primers.

In various embodiments, the single cell analysis workflow disclosed herein involving the implementation of digestible primers followed by digestion of the digestible primers achieves at least a 2-fold increase in percentage of reads with a valid barcode after trimming in comparison to a workflow process that implements oligo dT primers as opposed to digestible primers. In various embodiments, the single cell analysis workflow disclosed herein involving the implementation of digestible primers followed by digestion of the digestible primers achieves at least a 3-fold increase, at least a 4-fold increase, or at least a 5-fold increase in percentage of reads with a valid barcode after trimming in comparison to a workflow process that implements oligo dT primers as opposed to digestible primers.

In various embodiments, the single cell analysis workflow disclosed herein involving the implementation of digestible primers followed by digestion of the digestible primers achieves at least a 2-fold increase in percentage of oligo dT/dU reads after trimming in comparison to a workflow process that implements oligo dT primers as opposed to digestible primers. In various embodiments, the single cell analysis workflow disclosed herein involving the implementation of digestible primers followed by digestion of the digestible primers achieves at least a 3-fold increase, at least a 4-fold increase, or at least a 5-fold increase in percentage of oligo dT/dU reads after trimming in comparison to a workflow process that implements oligo dT primers as opposed to digestible primers.

In various embodiments, the single cell analysis workflow disclosed herein involving the implementation of digestible primers followed by digestion of the digestible primers achieves at least a 2-fold increase in percentage of reads with the forward primer after trimming in comparison to a workflow process that implements oligo dT primers as opposed to digestible primers. In various embodiments, the single cell analysis workflow disclosed herein involving the implementation of digestible primers followed by digestion of the digestible primers achieves at least a 3-fold increase, at least a 4-fold increase, or at least a 5-fold increase in percentage of reads with the forward primer in comparison to a workflow process that implements oligo dT primers as opposed to digestible primers.

Example Processing of RNA and DNA Using Digestible Primers

Targeted DNA/RNA Sequencing

Embodiments disclosed herein refer to a single cell workflow process for targeted DNA/RNA sequencing using digestible primers. In various embodiments, the targeted DNA/RNA sequencing workflow uses digestible primers having one or more ribonucleotide nucleobases, hereafter referred to as a ribonucleotide primer. In various embodiments, a ribonucleotide primer comprises a combination of deoxyribonucleotides and ribonucleotides. Additionally, the targeted DNA/RNA sequencing workflow implements an RNase (e.g., RNaseH or RNaseA) to digest the ribonucleotide primers. In various embodiments, the ribonucleotide primers are provided in the reagents (e.g., reagents 120 in FIG. 1B) and the RNase is provided in the reaction mixture (e.g., reaction mixture 140 in FIG. 1B). In various embodiments, the reaction mixture further includes additional primers for nucleic acid amplification. Additional primers can include forward DNA primer (which hybridizes to cDNA) and a primer pair (which hybridizes to gDNA). In various embodiments, the RNase digests the ribonucleotide primers after a first cycle of nucleic acid amplification. In various embodiments, the reaction mixture further includes a barcode sequence. Thus, the barcode sequence can be incorporated into amplicons through the nucleic acid amplification process.

FIG. 4A depicts the processing of RNA and gDNA in a first droplet, in accordance with an embodiment for targeted transcriptome sequencing. FIG. 4A depicts the step of analyte release 165 described in FIG. 1B and generally, the progression in FIGS. 3A-3C. Thus, in some embodiments, the steps depicted in FIG. 4A occur within a first droplet.

Within the droplet, a RNA transcript 410 is primed using a digestible primer 405. As shown in FIG. 4A, the digestible primer 405 is a reverse primer that hybridizes with a complementary portion of the RNA transcript 410. In various embodiments, the digestible primer 405 is a gene specific primer that targets a complementary portion of the RNA transcript that is transcribed from the gene. In this scenario, the digestible primer 405 is a ribonucleotide primer that contains a mixture of deoxyribonucleotide nucleobases and ribonucleotide bases. Various embodiments of ribonucleotide primers are described in further detail below. In various embodiments, the digestible primer 405 further includes a read sequence (labeled as “32092” in FIG. 4A). In some embodiments, the digestible primer need not include the read sequence. Following priming of the RNA transcript 410 using the digestible primer 405, reverse transcriptase extends the complementary strand to generate a cDNA strand 420 including the digestible primer. Furthermore, genomic DNA (gDNA) 425 is released by exposing chromatin to proteases, such as proteinase K.

FIG. 4B depicts the amplification and barcoding of nucleic acids derived from RNA and gDNA, in accordance with the embodiment shown in FIG. 4A. FIG. 4B depicts the step of cell barcoding 170 and target amplification 175 described in FIG. 1B. Thus, in some embodiments, the steps depicted in FIG. 4B occur within a second droplet.

Here, the top of FIG. 4B depicts the cDNA strand 420 and gDNA 425, each of which can be primed with respective primers that are added into the droplet as the reaction mixture. For example, a forward primer 430 can hybridize with a complementary region of the cDNA strand 420. In various embodiments, the forward primer 430 is a gene specific primer. In various embodiments, the forward primer 430 further includes a constant region (referred to as “seq8F” in FIG. 4B). Furthermore, a forward primer 435A and reverse primer 435B pair can hybridize with the gDNA. In various embodiments, the forward primer 435A and reverse primer 435B are gene specific primers that target a region of the gDNA corresponding to a specific gene. In various embodiments, the reverse primer further comprises a read sequence (labeled as “Read 2” in FIG. 4B).

Complementary strands for the cDNA strand 420 and gDNA 425 are synthesized off of the respective primers (e.g., forward primer 430 and primer pair 435A and 435B). As shown in the middle panel of FIG. 4B, complementary strand 426 is synthesized from the cDNA strand 420. Here, the complementary strand 426 further includes a sequence 428 that is complementary to the digestible primer 405. The sequence 428 comprises deoxyribonucleotide nucleobases and does not comprise ribonucleotide nucleobases.

The digestible primer 405 is digested from the original cDNA strand 405 to prevent the digestible primer 405 from participating in subsequent reactions (e.g., subsequent nucleic acid amplification reactions). Here, the digestible primer is exposed to a RNase (e.g., RNaseH or RNaseA that is present in the reaction mixture), which digests and removes the digestible primer due to the presence of ribonucleotide nucleobases in the digestible primer. Notably, in the middle panel of FIG. 4B, the enzyme 440 (e.g., RNase) acts to digest the digestible primer 405, but not the sequence 428 that is complementary to the digestible primer 405 because of the lack of ribonucleotide nucleobases in the sequence 428. Although not shown, the enzyme also digests the digestible primers that may have formed primer byproducts and/or misprimed nucleic acids (e.g., digestible primers that primed the genomic DNA).

The bottom panel depicts the later cycles of nucleic acid amplification in which the digestible primer 405 is no longer present. Here, additional amplicons (e.g., amplicon 460 derived from the cDNA strand and amplicon 470 derived from gDNA) are generated. Additionally, barcodes can be incorporated into the amplicons. For example, a barcode sequence may include a constant region (labeled as “seq8F”) that hybridizes with a constant region of the forward primer 430 or the constant region of the forward primer 435A. Therefore, nucleic acid extension generates a new amplicon that incorporates the barcode sequence.

Nested Targeted DNA/RNA Sequencing

Embodiments disclosed herein refer to a single cell workflow process for nested targeted DNA/RNA sequencing using digestible primers. In various embodiments, the nested targeted DNA/RNA sequencing workflow uses digestible primers having one or more ribonucleotide nucleobases (e.g., ribonucleotide primer) or digestible uracil primers. In some embodiments where the digestible primers are ribonucleotide primers, the nested targeting DNA/RNA sequencing workflow implements RNase (e.g., RNaseH or RNaseA) to digest the ribonucleotide primers. In some embodiments where the digestible primers are uracil primers, the nested targeting DNA/RNA sequencing workflow implements uracil-DNA glycosylase (UDG) to digest the uracil primers. In various embodiments, the digestible primers are provided in the reagents (e.g., reagents 120 in FIG. 1B) and the RNase or UDG is provided in the reaction mixture (e.g., reaction mixture 140 in FIG. 1B). In various embodiments, the RNase digests the ribonucleotide primers or UDG digests uracil primers prior to a first cycle of nucleic acid amplification. In various embodiments, the reaction mixture further includes additional primers for nucleic acid amplification. Additional primers can include forward and reverse primers for the cDNA. Additional primers can include a primer pair for the gDNA. In various embodiments, the reaction mixture further includes a barcode sequence. Thus, the barcode sequence can be incorporated into amplicons through the nucleic acid amplification process.

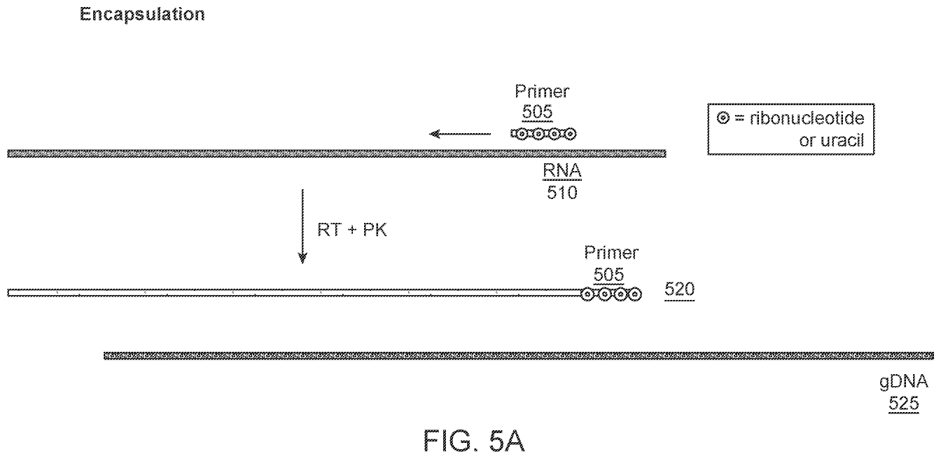

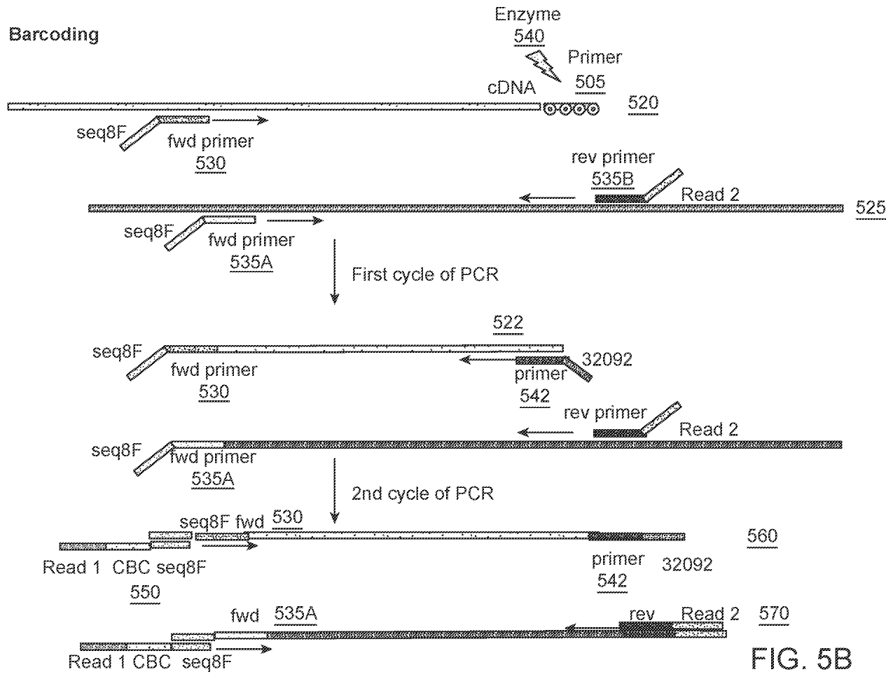

FIG. 5A depicts the processing of RNA and gDNA in a first droplet, in accordance with an embodiment for nested targeted transcriptome sequencing. FIG. 5A depicts the step of analyte release 165 described in FIG. 1B and generally, the progression in FIGS. 3A-3C. Thus, in some embodiments, the steps depicted in FIG. 5A occur within a first droplet.

Within the droplet, a RNA transcript 510 is primed using a digestible primer 505. As shown in FIG. 5A, the digestible primer 505 is a reverse primer that hybridizes with a complementary portion of the RNA transcript 510. In various embodiments, the digestible primer 505 is a gene specific primer that targets a complementary portion of the RNA transcript 510 that is transcribed from the gene. In some embodiments, the digestible primer 505 is a ribonucleotide primer that contains a mixture of deoxyribonucleotide nucleobases and ribonucleotide bases. In some embodiments, the digestible primer 505 contains is a uracil primer. In various embodiments, the uracil primer contains one or more uracil nucleobases. In particular embodiments, the digestible primer 505 contains 3 or more consecutive uracil nucleobases. Various embodiments of ribonucleotide primers and digestible uracil primers are described in further detail below. Following priming of the RNA transcript 510 using the digestible primer 505, reverse transcriptase that is provided as part of the reagents extends the complementary strand to generate a cDNA strand 520 including the digestible primer. Furthermore, genomic DNA (gDNA) 525 is released by exposing chromatin to proteases, such as proteinase K.

FIG. 5B depicts the amplification and barcoding of nucleic acids derived from RNA and gDNA, in accordance with the embodiment shown in FIG. 5A. FIG. 5B depicts the step of cell barcoding 170 and target amplification 175 described in FIG. 1B. Thus, in some embodiments, the steps depicted in FIG. 5B occur within a second droplet.

Here, the top of FIG. 5B depicts the cDNA strand 520 and gDNA 525, each of which can be primed with respective primers that are added into the droplet as the reaction mixture. For example, a forward primer 530 can hybridize with a complementary region of the cDNA strand 520. In various embodiments, the forward primer 530 is a gene specific primer. In various embodiments, the forward primer 530 further includes a constant region (referred to as “seq8F” in FIG. 5B). Furthermore, a forward primer 535A and reverse primer 535B pair can hybridize with the gDNA 525. In various embodiments, the forward primer 535A and reverse primer 535B are gene specific primers that target a region of the gDNA corresponding to a specific gene. In various embodiments, the forward primer 535A includes a constant region (referred to as “seq8F” in FIG. 5B). In various embodiments, the reverse primer 535B further comprises a read sequence (labeled as “Read 2” in FIG. 5B).

As shown in FIG. 5B, the digestible primer 505 in the cDNA strand 520 is digested. Generally, the digestible primer 505 is digested prior to the first cycle of nucleic acid amplification. In various embodiments, the digestible primer 505 is digested prior to the synthesis of the complementary cDNA strand (e.g., cDNA 522) such that the complementary cDNA strand lacks a sequence that is complementary to the digestible primer 505.

In various embodiments, the digestible primer 505 is digested using an enzyme 540 that is provided in the reaction mix. In various embodiments, the enzyme 540 is a RNase (e.g., RNaseH or RNaseA). For example, the digestible primer 505 is a ribonucleotide primer and therefore, can be digested by RNase. In various embodiments, the enzyme 540 is uracil-DNA glycosylase (UDG). For example, the digestible primer 505 is a uracil primer and therefore, can be digested by UDG. Although not shown, the enzyme 540 also digests the digestible primers that may have formed primer byproducts and/or misprimed nucleic acids (e.g., digestible primers that primed the genomic DNA). Altogether, the presence of digestible primer 505 is reduced or eliminated following exposure to the enzyme 540 and therefore, cannot participate in the subsequent nucleic acid amplification reactions.

Complementary strands for each of the cDNA strand 520 and gDNA 525 are synthesized off of the respective primers (e.g., forward primer 530 and primer pair 535A and 535B). As shown in the middle panel of FIG. 4B, complementary strand 522 is synthesized. Here, the complementary strand 522 does not include the digestible primer 505, nor does it include a sequence complementary to the digestible primer 505.

A primer, referred to as primer 542 in FIG. 5B, hybridizes with the complementary strand 522. Here, the primer 542 is different from the previously digested digestible primer (hence the “nested” nomenclature). Here, the primer 542 can be provided from the reaction mix. In various embodiments, the primer 542 is a reverse primer. In various embodiments, the primer 542 is a gene specific primer. Generally, the primer 542 enables the subsequent cycles of nucleic acid amplification.

The bottom panel depicts the later cycles of nucleic acid amplification in which the digestible primer 505 is not present. Here, additional amplicons (e.g., amplicon 560 derived from the cDNA strand and amplicon 570 derived from gDNA) are generated. Additionally, a barcode sequence 550 can be incorporated into the amplicons due to the nucleic acid amplification reaction. For example, a barcode sequence 550 may include a constant region (labeled as “seq8F”) that hybridizes with a constant region of the forward primer 530 or the constant region of the forward primer 535A. Therefore, nucleic acid extension generates a new amplicon that incorporates the barcode sequence.

Whole Transcriptome Sequencing

Embodiments disclosed herein refer to a single cell workflow process for whole transcriptome sequencing using digestible primers. In various embodiments, the whole transcriptome sequencing workflow uses digestible primers having either a repeating deoxyuridine sequence (e.g., oligo dUracil or oligo dU), or having a repeating ribouridine sequence (e.g., oligo rUracil or oligo rU). In some embodiments where the digestible primers are oligo dU primers, the whole transcriptome sequencing workflow implements UDG to digest the oligo dU primers. In some embodiments where the digestible primers are oligo rU primers, the whole transcriptome sequencing workflow implements RNaseH to digest the oligo rU primers.

In various embodiments, the digestible primers are provided in the reagents (e.g., reagents 120 in FIG. 1B). In various embodiments, the RNaseH or UDG is also provided in the reagents (e.g., reagents 120 in FIG. 1B). In various embodiments, the RNaseH or UDG is provided in the reaction mixture (e.g., reaction mixture 140 in FIG. 1B). In various embodiments, the RNaseH digests the ribonucleotide primers or the UDG digests uracil primers within a first droplet (e.g., droplet formed during cell encapsulation 160 in FIG. 1B). In various embodiments, the RNaseH digests the ribonucleotide primers or the UDG digests uracil primers within a second droplet (e.g., droplet formed during cell barcoding 170 in FIG. 1B). In various embodiments, the RNaseH digests the ribonucleotide primers or the UDG digests uracil primers prior to a first cycle of nucleic acid amplification. In various embodiments, the reaction mixture further includes additional primers for nucleic acid amplification. Additional primers can include forward and reverse primers for the cDNA. Additional primers can include a primer pair for the gDNA. In various embodiments, the reaction mixture further includes a barcode sequence. Thus, the barcode sequence can be incorporated into amplicons through the nucleic acid amplification process.

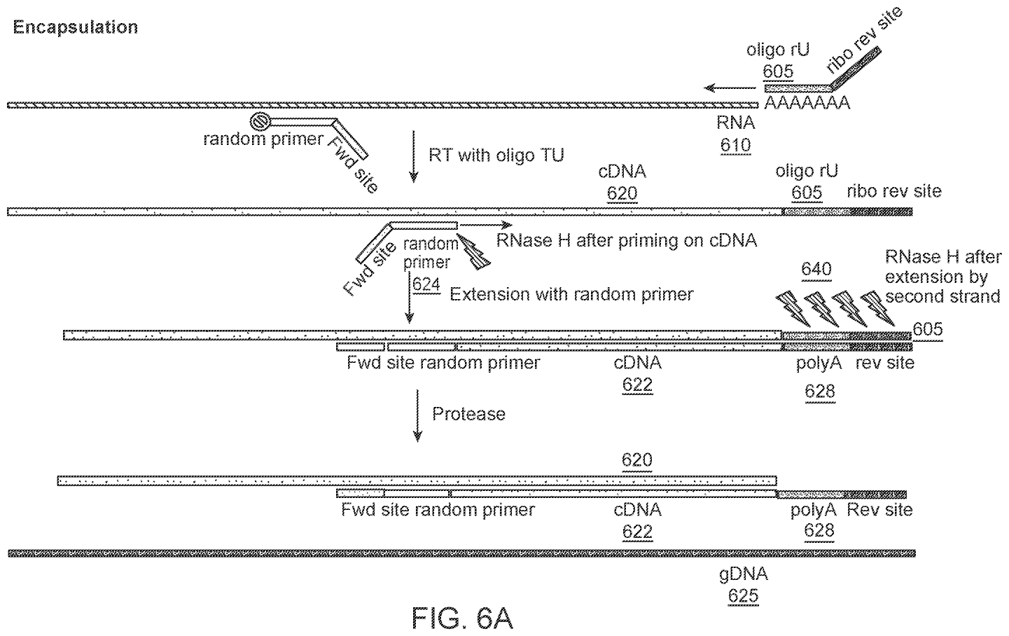

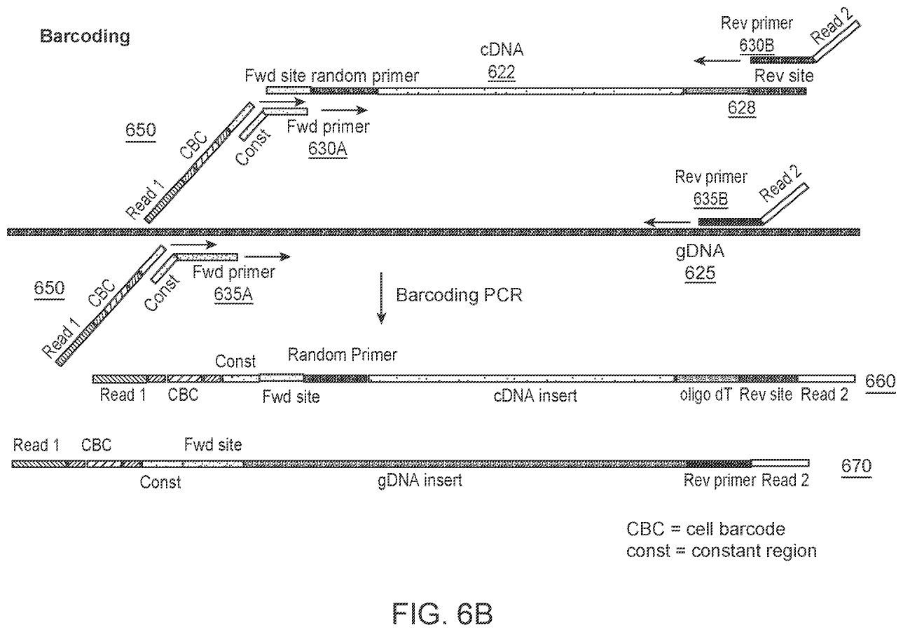

FIG. 6A depicts the processing of RNA and gDNA in a first droplet, in accordance with a first embodiment for whole transcriptome sequencing. This first embodiment uses a oligo rU digestible primer (e.g., primer with repeating ribouridine sequence). FIG. 6A depicts the step of analyte release 165 described in FIG. 1B and generally, the progression in FIGS. 3A-3C. Thus, in some embodiments, the steps depicted in FIG. 6A occur within a first droplet.

Within the droplet, a RNA transcript 610 is primed using a digestible primer 605 (labeled as an “oligo rU” primer 605). As shown in FIG. 6A, the digestible primer 605 is a reverse primer that hybridizes with a complementary portion of the RNA transcript 610. In various embodiments, the digestible primer 605 is a universal primer that targets a complementary portion of the RNA transcript 610. For example, the digestible primer 605 is an oligo rU primer (e.g., primer with repeating ribouridine sequence) that is complementary to a polyA tail of the RNA transcript 610. In various embodiments, the digestible primer 605 further includes a constant region (referred to in FIG. 6A as “ribo rev site”).

Following priming of the RNA transcript 610 using the digestible primer 605, reverse transcriptase that is provided as part of the reagents extends the complementary strand to generate a cDNA strand 620 including the digestible primer 605. The cDNA strand 620 including the digestible primer 605 is primed using a random primer 624. The random primer 624 is complementary to a region of the cDNA strand 620. In various embodiments, as shown in FIG. 6A, the random primer 624 further includes a constant region (referred to in FIG. 6A as “fwd site”). In various embodiments, the random primer 624 includes one or more ribonucleotide nucleobases. For example, the random primer 624 can include one or more ribonucleotide nucleobases on the 3′ end such that the random primer only extends on cDNA and not on RNA. Therefore, after priming on the cDNA, the random primer can be exposed to RNase (e.g., RNaseH) to enable extension along the cDNA.

The random primer 624 is extended, thereby generating a complementary cDNA strand 622 (complementary to cDNA strand 620). Here, complementary cDNA strand 622 includes a sequence 628 that is complementary to the digestible primer 605. For example, if the digestible primer 605 is an oligo rU primer, the sequence 628 is a polyA sequence.

After extension and generation of the complementary cDNA strand 622, the digestible primer 605 in the cDNA strand 620 is digested. In various embodiments, the digestible primer 605 is digested using an enzyme 640 that is provided in the reagents. In various embodiments, the enzyme is a RNaseH. For example, the digestible primer 605 is an oligo rU primer and therefore, can be digested by RNaseH. Although not shown, the enzyme 640 also digests the digestible primers that may have formed primer byproducts and/or misprimed nucleic acids (e.g., digestible primers that primed the genomic DNA). Altogether, the presence of digestible primer 605 is reduced or completely eliminated following exposure to the enzyme 640 and therefore, cannot participate in the subsequent nucleic acid amplification reactions.

The bottom panel of FIG. 6A shows the resulting cDNA strand 620 that no longer includes the digestible primer 605 as well as the complementary cDNA strand 622 that includes the sequence 628. Furthermore, genomic DNA (gDNA) 525 is released by exposing chromatin to proteases, such as proteinase K.

FIG. 6B depicts the amplification and barcoding of nucleic acids derived from RNA and gDNA, in accordance with the embodiment shown in FIG. 6A. FIG. 6B depicts the step of cell barcoding 170 and target amplification 175 described in FIG. 1B. Thus, in some embodiments, the steps depicted in FIG. 6B occur within a second droplet.

Here, the top panel of FIG. 6B depicts the complementary cDNA strand 622 with sequence 628 and gDNA 625, each of which can be primed with respective primers that are added into the droplet as the reaction mixture. For example, a primer pair (e.g., forward primer 630A and reverse primer 630B) can hybridize with complementary constant regions of the complementary cDNA strand 622. Specifically, the forward primer 630A hybridizes with the constant region of the random primer 624 whereas the reverse primer 630B hybridizes with a constant region of the sequence 628. As shown in FIG. 6B, the reverse primer 630B includes a read sequence, referred to as “Read 2.” The forward primer 630A may further include a constant region that enables hybridization to a complementary constant region of a barcode sequence 650, thereby enabling incorporation of the barcode sequence 650.

Referring now to the gDNA 625, a primer pair (e.g., forward primer 635A and reverse primer 635B) can hybridize with complementary regions of the gDNA 625. In various embodiments, the forward primer 635A and reverse primer 635B are gene specific primers. As shown in FIG. 6B, the reverse primer 635B includes a read sequence, referred to as “Read 2.” The forward primer 635A may further include a constant region that enables hybridization to a complementary constant region of a barcode sequence 650, thereby enabling incorporation of the barcode sequence 650.

Subsequent cycles of nucleic acid amplification (in which digestible primers are not present) generate amplicon 660 derived from the cDNA 622 and amplicon 670 derived from the gDNA 625.

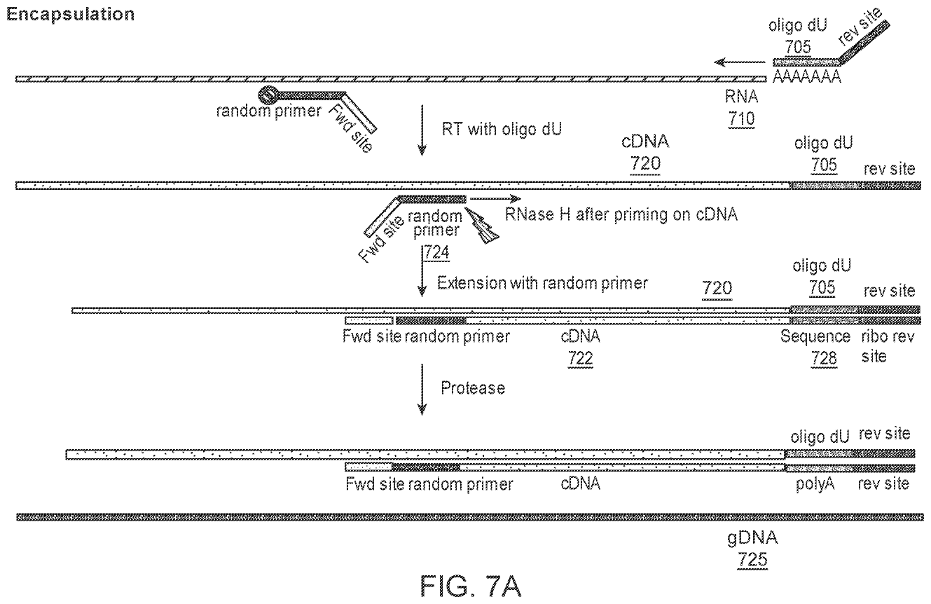

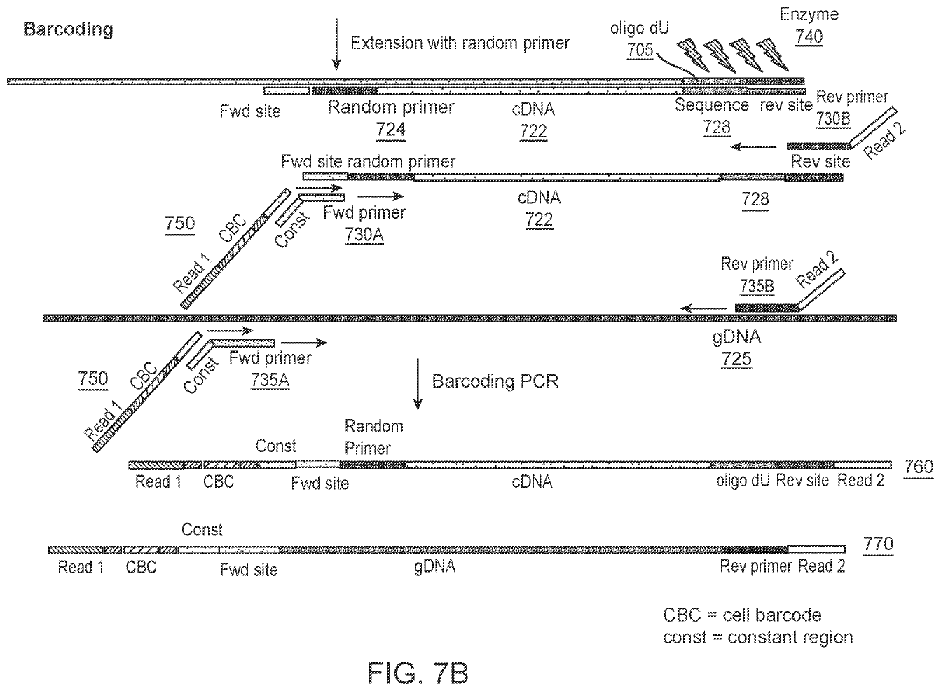

FIG. 7A depicts the processing of RNA and gDNA in a first droplet, in accordance with a second embodiment for whole transcriptome sequencing. This second embodiment uses a oligo dU digestible primer (e.g., primer with repeating deoxyuridine sequence). FIG. 7A depicts the step of analyte release 165 described in FIG. 1B and generally, the progression in FIGS. 3A-3C. Thus, in some embodiments, the steps depicted in FIG. 7A occur within a first droplet.

Within the droplet, a RNA transcript 710 is primed using a digestible primer 705. As shown in FIG. 7A, the digestible primer 705 is a reverse primer that hybridizes with a complementary portion of the RNA transcript 710. In various embodiments, the digestible primer 705 is a universal primer that targets a complementary portion of the RNA transcript 710. For example, the digestible primer 705 is an oligo dU primer (e.g., primer with repeating deoxyuridine sequence) that is complementary to a polyA tail of the RNA transcript 710. In various embodiments, the digestible primer 705 further includes a constant region (referred to in FIG. 7A as “rev site”).

Following priming of the RNA transcript 710 using the digestible primer 705, reverse transcriptase that is provided as part of the reagents extends the complementary strand to generate a cDNA strand 720 including the digestible primer 705. The cDNA strand 720 including the digestible primer 705 is primed using a random primer 724. The random primer 724 is complementary to a region of the cDNA strand 720. In various embodiments, as shown in FIG. 7A, the random primer 724 further includes a constant region (referred to in FIG. 7A as “fwd site”). In various embodiments, the random primer 724 includes one or more ribonucleotide nucleobases. For example, the random primer 724 can include one or more ribonucleotide nucleobases on the 3′ end such that the random primer only extends on cDNA and not on RNA. Therefore, after priming on the cDNA, the random primer 724 can be exposed to RNase (e.g., RNaseH) to enable extension along the cDNA.

The random primer 724 is extended, thereby generating a complementary cDNA strand 722 (complementary to cDNA strand 720). Here, complementary cDNA strand 722 includes a sequence 728 that is complementary to the digestible primer 705. For example, if the digestible primer 705 is an oligo dU primer, the sequence 628 is a polyA sequence.

Furthermore, genomic DNA (gDNA) 525 is released by exposing chromatin to proteases, such as proteinase K.

FIG. 7B depicts the amplification and barcoding of nucleic acids derived from RNA and gDNA, in accordance with the embodiment shown in FIG. 7A. FIG. 7B depicts the step of cell barcoding 170 and target amplification 175 described in FIG. 1B. Thus, in some embodiments, the steps depicted in FIG. 7B occur within a second droplet.

Here, the top panel of FIG. 7B depicts the double-stranded cDNA (including the cDNA strand 720 and complementary cDNA strand 722). The digestible primer 705 in the cDNA strand 720 is digested. In various embodiments, the digestible primer 705 is digested using an enzyme 740 that is provided in the reaction mix. In various embodiments, the enzyme is UDG. For example, the digestible primer 705 is an oligo dU primer and therefore, can be digested by UDG. Thus, the presence of digestible primer 705 is reduced and/or eliminated such that the digestible primer 705 does not participate in subsequent nucleic acid amplification reactions.

The complementary cDNA strand 722 including the sequence 728 is primed. For example, a primer pair (e.g., forward primer 730A and reverse primer 730B) can hybridize with complementary constant regions of the complementary cDNA strand 722. Specifically, the forward primer 730A hybridizes with the constant region of the random primer 724 whereas the reverse primer 730B hybridizes with a constant region of the sequence 728. As shown in FIG. 7B, the reverse primer 730B includes a read sequence, referred to as “Read 2.” The forward primer 730A may further include a constant region that enables hybridization to a complementary constant region of a barcode sequence 750, thereby enabling incorporation of the barcode sequence 750.

Referring now to the gDNA 725, a primer pair (e.g., forward primer 735A and reverse primer 735B) can hybridize with complementary regions of the gDNA 725. In various embodiments, the forward primer 735A and reverse primer 735B are gene specific primers. As shown in FIG. 7B, the reverse primer 735B includes a read sequence, referred to as “Read 2.” The forward primer 735A may further include a constant region that enables hybridization to a complementary constant region of a barcode sequence 750, thereby enabling incorporation of the barcode sequence 750.

Subsequent cycles of nucleic acid amplification (in which digestible primers are not present) generate amplicon 760 derived from the cDNA 722 and amplicon 770 derived from the gDNA 725.

Barcodes and Barcoded Beads

Embodiments of the invention involve providing one or more barcode sequences for labeling analytes of a single cell during step 170 shown in FIG. 1B. The one or more barcode sequences are encapsulated in an emulsion with a cell lysate derived from a single cell. As such, the one or more barcodes label analytes of the cell, thereby enabling the subsequent determination that sequence reads derived from the analytes originated from the cell.

In various embodiments, a plurality of barcodes are added to an emulsion with a cell lysate. In various embodiments, the plurality of barcodes added to an emulsion includes at least 10², at least 10³, at least 10⁴, at least 10⁵, at least 10⁵, at least 10⁶, at least 10⁷, or at least 10⁸ barcodes. In various embodiments, the plurality of barcodes added to an emulsion have the same barcode sequence. In various embodiments, the plurality of barcodes added to an emulsion comprise a ‘unique identification sequence’ (UMI). A UMI is a nucleic acid having a sequence which can be used to identify and/or distinguish one or more first molecules to which the UMI is conjugated from one or more second molecules. UMIs are typically short, e.g., about 5 to 20 bases in length, and may be conjugated to one or more target molecules of interest or amplification products thereof. UMIs may be single or double stranded. In some embodiments, both a barcode sequence and a UMI are incorporated into a barcode. Generally, a UMI is used to distinguish between molecules of a similar type within a population or group, whereas a barcode sequence is used to distinguish between populations or groups of molecules that are derived from different cells. Thus, a UMI can be used to count or quantify numbers of particular molecules (e.g., quantify number of RNA transcripts). In some embodiments, where both a UMI and a barcode sequence are utilized, the UMI is shorter in sequence length than the barcode sequence. The use of barcodes is further described in U.S. patent application Ser. No. 15/940,850, which is hereby incorporated by reference in its entirety.

In some embodiments, the barcodes are single-stranded barcodes. Single-stranded barcodes can be generated using a number of techniques. For example, they can be generated by obtaining a plurality of DNA barcode molecules in which the sequences of the different molecules are at least partially different. These molecules can then be amplified so as to produce single stranded copies using, for instance, asymmetric PCR. Alternatively, the barcode molecules can be circularized and then subjected to rolling circle amplification. This will yield a product molecule in which the original DNA barcoded is concatenated numerous times as a single long molecule.

In some embodiments, circular barcode DNA containing a barcode sequence flanked by any number of constant sequences can be obtained by circularizing linear DNA. Primers that anneal to any constant sequence can initiate rolling circle amplification by the use of a strand displacing polymerase (such as Phi29 polymerase), generating long linear concatemers of barcode DNA.

In various embodiments, barcodes can be linked to a primer sequence that enables the barcode to label a target nucleic acid. In one embodiment, the barcode is linked to a forward primer sequence. In various embodiments, the forward primer sequence is a gene specific primer that hybridizes with a forward target of a nucleic acid. In various embodiments, the forward primer sequence is a constant region, such as a PCR handle, that hybridizes with a complementary sequence attached to a gene specific primer. The complementary sequence attached to a gene specific primer can be provided in the reaction mixture (e.g., reaction mixture 140 in FIG. 1B). Including a constant forward primer sequence on barcodes may be preferable as the barcodes can have the same forward primer and need not be individually designed to be linked to gene specific forward primers.

In various embodiments, barcodes can releasably attached to a support structure, such as a bead. Therefore, a single bead with multiple copies of barcodes can be partitioned into an emulsion with a cell lysate, thereby enabling labeling of analytes of the cell lysate with the barcodes of the bead. Example beads include solid beads (e.g., silica beads), polymeric beads, or hydrogel beads (e.g., polyacrylamide, agarose, or alginate beads). Beads can be synthesized using a variety of techniques. For example, using a mix-split technique, beads with many copies of the same, random barcode sequence can be synthesized. This can be accomplished by, for example, creating a plurality of beads including sites on which DNA can be synthesized. The beads can be divided into four collections and each mixed with a buffer that will add a base to it, such as an A, T, G, or C. By dividing the population into four subpopulations, each subpopulation can have one of the bases added to its surface. This reaction can be accomplished in such a way that only a single base is added and no further bases are added. The beads from all four subpopulations can be combined and mixed together, and divided into four populations a second time. In this division step, the beads from the previous four populations may be mixed together randomly. They can then be added to the four different solutions, adding another, random base on the surface of each bead. This process can be repeated to generate sequences on the surface of the bead of a length approximately equal to the number of times that the population is split and mixed. If this was done 10 times, for example, the result would be a population of beads in which each bead has many copies of the same random 10-base sequence synthesized on its surface. The sequence on each bead would be determined by the particular sequence of reactors it ended up in through each mix-split cycle. Additional details of example beads and their synthesis is described in International Application No. PCT/US2016/016444, which is hereby incorporated by reference in its entirety.

Reagents

Embodiments described herein include the encapsulation of a cell with reagents within an emulsion. In various embodiments, the reagents interact with the encapsulated cell under conditions in which the cell is lysed, thereby releasing target analytes of the cell. The reagents can further interact with target analytes to prepare for subsequent barcoding and/or amplification.

In various embodiments, the reagents include one or more lysing agents that cause the cell to lyse. Examples of lysing agents include detergents such as Triton X-100, Nonidet P-40 (NP40) as well as cytotoxins. In various embodiments, the reagents further include agents that interact with target analytes that are released from a single cell. One example of such an agent includes reverse transcriptase which reverse transcribes messenger RNA transcripts released from the cell to generate corresponding cDNA.

In various embodiments, the reagents encapsulated with the cell include ddNTPs, inhibitors such as ribonuclease inhibitor, and stabilization agents such as dithothreitol (DTT). In various embodiments, the reagents further include proteases that assist in the lysing of the cell and/or accessing of genomic DNA. In various embodiments, proteases in the reagents can include any of proteinase K, pepsin, protease—subtilisin Carlsberg, protease type X-Bacillus thermoproteolyticus, or protease type XIII—Aspergillus Saitoi. In various embodiments, the reagents include deoxyribonucleotide triphosphate (dNTP) reagents including deoxyadenosine triphosphate, deoxycytosine triphosphate, deoxyguanine triphosphate, and deoxythymidine triphosphate.

In various embodiments, the reagents include agents that interact with target analytes that are released from a single cell. For example, the reagents include reverse transcriptase which reverse transcribes mRNA transcripts released from the cell to generate corresponding cDNA. As another example, the reagents include primers that hybridize with mRNA transcripts, thereby enabling the reverse transcription reaction to occur. In various embodiments, such primers are digestible primers that participate in the reverse transcription reaction, but are subsequently digested to prevent their participation in subsequent reactions.

In various embodiments, the reagents include agents for digesting the digestible primers. In such embodiments, the agents digest the digestible primers while in a droplet, such as a first droplet generated during the cell encapsulation step (step 160 in FIG. 1B). In various embodiments, agents for digesting the digestible primers are enzymes. In some embodiments, an agent for digesting the digestible primers is a RNaseH enzyme. In various embodiments, the reagents includes a concentration of at least 0.01 Units/μL of RNaseH enzyme. In various embodiments, the reagents includes at least a concentration of 0.05 Units/μL of RNaseH enzyme, at least a concentration of 0.1 Units/μL of RNaseH enzyme, at least a concentration of 0.2 Units/μL of RNaseH enzyme, at least a concentration of 0.3 Units/μL of RNaseH enzyme, at least a concentration of 0.4 Units/μL of RNaseH enzyme, at least a concentration of 0.5 Units/μL of RNaseH enzyme, at least a concentration of 0.6 Units/μL of RNaseH enzyme, at least a concentration of 0.7 Units/μL of RNaseH enzyme, at least a concentration of 0.8 Units/μL of RNaseH enzyme, at least a concentration of 0.9 Units/μL of RNaseH enzyme, at least a concentration of 1.0 Units/μL of RNaseH enzyme, at least a concentration of 2.0 Units/μL of RNaseH enzyme, at least a concentration of 4.0 Units/μL of RNaseH enzyme, at least a concentration of 8.0 Units/μL of RNaseH enzyme, at least a concentration of 15 Units/μL of RNaseH enzyme, at least a concentration of 50 Units/μL of RNaseH enzyme, at least a concentration of 100 Units/μL of RNaseH enzyme, at least a concentration of 200 Units/μL of RNaseH enzyme, at least a concentration of 300 Units/μL of RNaseH enzyme, at least a concentration of 400 Units/μL of RNaseH enzyme, at least a concentration of 500 Units/μL of RNaseH enzyme, or at least a concentration of 1000 Units/μL of RNaseH enzyme. In various embodiments, the reagents include between 0.5 and 30 units of RNaseH enzyme. In various embodiments, the reagents include between 1 and 28 units of RNaseH enzyme. In various embodiments, the reagents include between 3 and 25 units of RNaseH enzyme. In various embodiments, the reagents include between 4 and 22 units of RNaseH enzyme. In various embodiments, the reagents include between 5 and 20 units of RNaseH enzyme. In various embodiments, the reagents include between 8 and 18 units of RNaseH enzyme. In various embodiments, the reagents include between 10 and 15 units of RNaseH enzyme. In various embodiments, the reagents include between 12 and 14 units of RNaseH enzyme.

Generally, the reagents do not include enzymes such as UDG or RNaseA because such enzymes will digest the digestible primers prior to their priming of the RNA transcript (for reverse transcription). Conversely, the reagents may include enzymes such as RNaseH because such enzymes will only digest the digestible primer once it is involved in an RNA-DNA duplex (e.g., after priming of the RNA transcript has occurred).

Reaction Mixture

As described herein, a reaction mixture is provided into an emulsion with a cell lysate (e.g., see cell barcoding step 170 in FIG. 1B). Generally, the reaction mixture includes reactants sufficient for performing a reaction, such as nucleic acid amplification, on analytes of the cell lysate.

In various embodiments, the reaction mixture includes primers that are capable of acting as a point of initiation of synthesis along a complementary strand when placed under conditions in which synthesis of a primer extension product which is complementary to a nucleic acid strand is catalyzed. In various embodiments, the reaction mixture includes the four different deoxyribonucleoside triphosphates (adenosine, guanine, cytosine, and thymine). In various embodiments, the reaction mixture includes enzymes for nucleic acid amplification. Examples of enzymes for nucleic acid amplification include DNA polymerase, thermostable polymerases for thermal cycled amplification, or polymerases for multiple-displacement amplification for isothermal amplification. Other, less common forms of amplification may also be applied, such as amplification using DNA-dependent RNA polymerases to create multiple copies of RNA from the original DNA target which themselves can be converted back into DNA, resulting in, in essence, amplification of the target. Living organisms can also be used to amplify the target by, for example, transforming the targets into the organism which can then be allowed or induced to copy the targets with or without replication of the organisms.

In various embodiments, the reagents include deoxyribonucleotide triphosphate (dNTP) reagents including deoxyadenosine triphosphate, deoxycytosine triphosphate, deoxyguanine triphosphate, and deoxythymidine triphosphate.

The extent of nucleic amplification can be controlled by modulating the concentration of the reactants in the reaction mixture. In some instances, this is useful for fine tuning of the reactions in which the amplified products are used.

In various embodiments, the reaction mixture include agents for digesting the digestible primers. In various embodiments, agents for digesting the digestible primers are enzymes. In such embodiments, the agents digest the digestible primers while in a droplet, such as a second droplet generated during the barcoding step (step 170 in FIG. 1B). The reaction mixture can include enzymes selected from any of UDG, RNaseH, or RNaseA. Here in the second droplet, the digestible primers have already primed the RNA transcript and reverse transcription has occurred. Therefore, providing any of these enzymes in the second droplet enables the digestion of the digestible primers after they participated in the reverse transcription reaction.

In some embodiments, an agent for digesting the digestible primers is an uracil-DNA glycosylase (UDG) enzyme. In various embodiments, the reagents includes a concentration of at least 0.01 Units/μL of UDG enzyme. In various embodiments, the reaction mixture includes at least a concentration of 0.05 Units/μL of UDG enzyme, at least a concentration of 0.1 Units/μL of UDG enzyme, at least a concentration of 0.2 Units/μL of UDG enzyme, at least a concentration of 0.3 Units/μL of UDG enzyme, at least a concentration of 0.4 Units/μL of UDG enzyme, at least a concentration of 0.5 Units/μL of UDG enzyme, at least a concentration of 0.6 Units/μL of UDG enzyme, at least a concentration of 0.7 Units/μL of UDG enzyme, at least a concentration of 0.8 Units/μL of UDG enzyme, at least a concentration of 0.9 Units/μL of UDG enzyme, at least a concentration of 1.0 Units/μL of UDG enzyme, at least a concentration of 2.0 Units/μL of UDG enzyme, at least a concentration of 4.0 Units/μL of UDG enzyme, at least a concentration of 8.0 Units/μL of UDG enzyme, at least a concentration of 15 Units/μL of UDG enzyme, at least a concentration of 50 Units/μL of UDG enzyme, at least a concentration of 100 Units/μL of UDG enzyme, at least a concentration of 200 Units/μL of UDG enzyme, at least a concentration of 300 Units/μL of UDG enzyme, at least a concentration of 400 Units/μL of UDG enzyme, at least a concentration of 500 Units/μL of UDG enzyme, or at least a concentration of 1000 Units/μL of UDG enzyme. In various embodiments, the reaction mixture include between 0.5 and 30 units of UDG enzyme. In various embodiments, the reaction mixture include between 1 and 28 units of UDG enzyme. In various embodiments, the reaction mixture include between 3 and 25 units of UDG enzyme. In various embodiments, the reaction mixture include between 4 and 22 units of UDG enzyme. In various embodiments, the reaction mixture include between 5 and 20 units of UDG enzyme. In various embodiments, the reaction mixture include between 8 and 18 units of UDG enzyme. In various embodiments, the reaction mixture include between 10 and 15 units of UDG enzyme. In various embodiments, the reaction mixture include between 12 and 14 units of UDG enzyme.

In some embodiments, an agent for digesting the digestible primers is a RNaseH enzyme. In various embodiments, the reaction mixture includes a concentration of at least 0.01 Units/μL of RNaseH enzyme. In various embodiments, the reaction mixture includes at least a concentration of 0.05 Units/μL of RNaseH enzyme, at least a concentration of 0.1 Units/μL of RNaseH enzyme, at least a concentration of 0.2 Units/μL of RNaseH enzyme, at least a concentration of 0.3 Units/μL of RNaseH enzyme, at least a concentration of 0.4 Units/μL of RNaseH enzyme, at least a concentration of 0.5 Units/μL of RNaseH enzyme, at least a concentration of 0.6 Units/μL of RNaseH enzyme, at least a concentration of 0.7 Units/μL of RNaseH enzyme, at least a concentration of 0.8 Units/μL of RNaseH enzyme, at least a concentration of 0.9 Units/μL of RNaseH enzyme, at least a concentration of 1.0 Units/μL of RNaseH enzyme, at least a concentration of 2.0 Units/μL of RNaseH enzyme, at least a concentration of 4.0 Units/μL of RNaseH enzyme, at least a concentration of 8.0 Units/μL of RNaseH enzyme, at least a concentration of 15 Units/μL of RNaseH enzyme, at least a concentration of 50 Units/μL of RNaseH enzyme, at least a concentration of 100 Units/μL of RNaseH enzyme, at least a concentration of 200 Units/μL of RNaseH enzyme, at least a concentration of 300 Units/μL of RNaseH enzyme, at least a concentration of 400 Units/μL of RNaseH enzyme, at least a concentration of 500 Units/μL of RNaseH enzyme, or at least a concentration of 1000 Units/μL of RNaseH enzyme. In various embodiments, the reaction mixture include between 0.5 and 30 units of RNaseH enzyme. In various embodiments, the reaction mixture include between 1 and 28 units of RNaseH enzyme. In various embodiments, the reaction mixture include between 3 and 25 units of RNaseH enzyme. In various embodiments, the reaction mixture include between 4 and 22 units of RNaseH enzyme. In various embodiments, the reaction mixture include between 5 and 20 units of RNaseH enzyme. In various embodiments, the reaction mixture include between 8 and 18 units of RNaseH enzyme. In various embodiments, the reaction mixture include between 10 and 15 units of RNaseH enzyme. In various embodiments, the reaction mixture include between 12 and 14 units of RNaseH enzyme.

In some embodiments, an agent for digesting the digestible primers is a RNaseA enzyme. In various embodiments, the reaction mixture includes a concentration of at least 0.01 Units/μL of RNaseA enzyme. In various embodiments, the reaction mixture includes at least a concentration of 0.05 Units/μL of RNaseA enzyme, at least a concentration of 0.1 Units/μL of RNaseA enzyme, at least a concentration of 0.2 Units/μL of RNaseA enzyme, at least a concentration of 0.3 Units/μL of RNaseA enzyme, at least a concentration of 0.4 Units/μL of RNaseA enzyme, at least a concentration of 0.5 Units/μL of RNaseA enzyme, at least a concentration of 0.6 Units/μL of RNaseA enzyme, at least a concentration of 0.7 Units/μL of RNaseA enzyme, at least a concentration of 0.8 Units/μL of RNaseA enzyme, at least a concentration of 0.9 Units/μL of RNaseA enzyme, at least a concentration of 1.0 Units/μL of RNaseA enzyme, at least a concentration of 2.0 Units/μL of RNaseA enzyme, at least a concentration of 4.0 Units/μL of RNaseA enzyme, at least a concentration of 8.0 Units/μL of RNaseA enzyme, at least a concentration of 15 Units/μL of RNaseA enzyme, at least a concentration of 50 Units/μL of RNaseA enzyme, at least a concentration of 100 Units/μL of RNaseA enzyme, at least a concentration of 200 Units/μL of RNaseA enzyme, at least a concentration of 300 Units/μL of RNaseA enzyme, at least a concentration of 400 Units/μL of RNaseA enzyme, at least a concentration of 500 Units/μL of RNaseA enzyme, or at least a concentration of 1000 Units/μL of RNaseA enzyme. In various embodiments, the reaction mixture include between 0.5 and 30 units of RNaseA enzyme. In various embodiments, the reaction mixture include between 1 and 28 units of RNaseA enzyme. In various embodiments, the reaction mixture include between 3 and 25 units of RNaseA enzyme. In various embodiments, the reaction mixture include between 4 and 22 units of RNaseA enzyme. In various embodiments, the reaction mixture include between 5 and 20 units of RNaseA enzyme. In various embodiments, the reaction mixture include between 8 and 18 units of RNaseA enzyme. In various embodiments, the reaction mixture include between 10 and 15 units of RNaseA enzyme. In various embodiments, the reaction mixture include between 12 and 14 units of RNaseA enzyme.

Primers

Embodiments of the invention described herein use primers to conduct the single-cell analysis. For example, primers are implemented during the workflow process shown in FIG. 1B. Primers can be used to prime (e.g., hybridize) with specific sequences of nucleic acids of interest, such that the nucleic acids of interest can be processed (e.g., reverse transcribed, barcoded, and/or amplified). Additionally, primers enable the identification of target regions following sequencing.

In various embodiments, primers described herein are between 5 and 50 nucleobases in length. In various embodiments, primers described herein are between 7 and 45 nucleobases in length. In various embodiments, primers described herein are between 10 and 40 nucleobases in length. In various embodiments, primers described herein are between 12 and 35 nucleobases in length. In various embodiments, primers described herein are between 15 and 32 nucleobases in length. In various embodiments, primers described herein are between 18 and 30 nucleobases in length. In various embodiments, primers described herein are between 18 and 25 nucleobases in length.

Referring again to FIG. 1B, in various embodiments, primers can be included in the reagents 120 that are encapsulated with the cell 110. In various embodiments, primers included in the reagents are useful for priming RNA transcripts and enabling reverse transcription of the RNA transcripts. In various embodiments, primers in the reagents 120 can include RNA primers for priming RNA and/or for priming genomic DNA. In various embodiments, the primers included in the reagents are digestible primers. Digestible primers can be digested at the appropriate time to ensure that subsequent reactions are not impacted by the presence of the digestible primers. In particular embodiments, digestible primers participate in a first reaction, such as a reverse transcriptase reaction, and are digested to prevent their participation in a second reaction, such as a nucleic acid amplification reaction.

In various embodiments, primers can be included in the reaction mixture 140 that is encapsulated with the cell lysate 130. In various embodiments, primers included in the reaction mixture are useful for priming nucleic acids (e.g., cDNA, gDNA, and/or amplicons of cDNA/gDNA) and enabling nucleic acid amplification of the nucleic acids. Such primers in the reaction mixture 140 can include cDNA primers for priming cDNA that have been reverse transcribed from RNA and/or DNA primers for priming genomic DNA and/or for priming products that have been generated from the genomic DNA. In various embodiments, primers of the reagents and primers of the reaction mixture form primer sets (e.g., forward primer and reverse primer) for a region of interest on a nucleic acid. In various embodiments, primers can be included in or linked with a barcode 145 that is encapsulated with the cell lysate 130. Further description and examples of primers that are used in a single-cell analysis workflow process is described in U.S. application Ser. No. 16/749,731, which is hereby incorporated by reference in its entirety.

In various embodiments, the number of primers in any of the reagents, the reaction mixture, or with barcodes may range from about 1 to about 500 or more, e.g., about 2 to 100 primers, about 2 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more.

For targeted nucleic acid (e.g., targeted DNA or targeted RNA) sequencing, primers in the reagents (e.g., reagents 120 in FIG. 1B) may include primers that are complementary to a target on a nucleic acid of interest (e.g., DNA or RNA). In various embodiments, primers in the reagents are gene-specific primers. In various embodiments, primers in the reagents are universal primers. Example universal primers include primers including at least 3 consecutive deoxythymidine nucleobases (e.g., oligo dT primer), at least 3 consecutive deoxyuridine sequences (e.g., oligo dU primer), or at least 3 consecutive ribouridine sequences (e.g., oligo rU primer).

In various embodiments, such primers in the reagents are reverse primers. In particular embodiments, primers in the reagents are only reverse primers and do not include forward primers. In various embodiments, for targeted nucleic acid (e.g., targeted DNA or targeted RNA) sequencing, primers in the reaction mixture (e.g., reaction mixture 140 in FIG. 1B) include forward primers that are complementary to a forward target on a nucleic acid of interest (e.g., RNA or gDNA). In particular embodiments, the reaction mixture includes forward primers that are complementary to a forward target on a cDNA strand (generated from a RNA transcript) and further includes forward primers that are complementary to a forward target on gDNA. In various embodiments, primers in the reaction mixture are gene-specific primers that target a forward target of a gene of interest.

The number of forward or reverse primers for genes of interest that are added may be from about one to 500, e.g., about 1 to 10 primers, about 10 to 20 primers, about 20 to 30 primers, about 30 to 40 primers, about 40 to 50 primers, about 50 to 60 primers, about 60 to 70 primers, about 70 to 80 primers, about 80 to 90 primers, about 90 to 100 primers, about 100 to 150 primers, about 150 to 200 primers, about 200 to 250 primers, about 250 to 300 primers, about 300 to 350 primers, about 350 to 400 primers, about 400 to 450 primers, about 450 to 500 primers, or about 500 primers or more. In various embodiments, genes of interest for either DNA-sequencing or RNA-sequencing include, but are not limited to: CCND3, CD44, CCND1, CD33, CDK6, CDK4, CDKN1B, CREB3L4, CDKN1A, CREBBP, CREB3L1, CREBS, CREB1, ELK1, FOS, FHL1, FASLG, GNG12, GSK3B, BAD, FOXO4, FOXO1, HIF1A, HSPB1, IKBKG, IRF9, BCL2, BCL2L11, MAP2K1 MAPK1, BCL2L1, MYB, NF1, NFKB1, MYC, PIK3CB, PIM1, PIAS1, PRKCB, PTEN, HSPA1A, HSPA2, IL2RB, IL2RA, SIRT1, NCL, RHOA, MCM4, NASP, SOS1, TCL1B, SOCS3, SOCS2, STAT4, STAT6, SRF, TP53, CASP9, CASP3, CASP8, UBB, MPRL16, MRPL21, FAM32A, ABCB7, PCBP1. EPS15, NRAS, RPS27A, AFF3, PAX3, CMTM6, RHOA, PIK3CA, MAP3K13, NSD1, PTPRK, CARD11, EGFR, EZH2, WRN, JAK2, GATA3, DKK1, POLA2, CCND1, ATM, ARHGEF12, KRAS, COL2A1, KMT2D, CLIP1, FLT3, BRCA2, BUB1B, PALB2, FANCA, NCOR1, ERBB2, KAT2A, RAB5C, METTL23, SRSF2, MFSD11, DNM2, CIC, BCR, MYH9, EP300, and SSX1.

For whole transcriptome RNA sequencing, in various embodiments, the primers of the reagents (e.g., reagents 120 in FIG. 1B) can include a random primer sequence. In various embodiments, the random primer hybridizes with a sequence of reverse transcribed cDNA, thereby enabling priming off of the cDNA. In various embodiments, the reagents 120 includes various different random primers that enables priming off of all or a majority of cDNA generated from mRNA transcripts across the transcriptome. This enables the processing and analysis of mRNA transcripts across the whole transcriptome. In various embodiments, a random primer comprises a sequence of 5 nucleobases. In various embodiments, a random primer comprises a sequence of 6 nucleobases. In various embodiments, a random primer comprises a sequence of 9 nucleobases. In various embodiments, a random primer comprises a sequence of at least 5 nucleobases. In various embodiments, a random primer comprises a sequence of at least 6 nucleobases. In various embodiments, a random primer comprises a sequence of at least 9 nucleobases. In various embodiments, a random primer comprises a sequence of at least 6 nucleobases, at least 7 nucleobases, at least 8 nucleobases, at least 9 nucleobases, at least 10 nucleobases, at least 11 nucleobases, at least 12 nucleobases, at least 13 nucleobases, at least 14 nucleobases, at least 15 nucleobases, at least 16 nucleobases, at least 17 nucleobases, at least 18 nucleobases, at least 19 nucleobases, at least 20 nucleobases, at least 21 nucleobases, at least 22 nucleobases, at least 23 nucleobases, at least 24 nucleobases, at least 25 nucleobases, at least 26 nucleobases, at least 27 nucleobases, at least 28 nucleobases, at least 29 nucleobases, at least 30 nucleobases, at least 31 nucleobases, at least 32 nucleobases, at least 33 nucleobases, at least 34 nucleobases, or at least 35 nucleobases.

In various embodiments, a random primer includes one or more ribonucleotide nucleobases. In some embodiments, the random primer 624 include one ribonucleotide nucleobase on the 3′ end. In some embodiments, the random primer 624 includes two ribonucleotide nucleobases on the 3′ end. In some embodiments, the random primer 624 includes three, four, five, six, seven, eight, nine, or ten ribonucleotide nucleobases on the 3′ end. The presence of ribonucleotide primers on the 3′ end of the random primer ensures that the random primer enables extension only on cDNA and not on RNA.

In various embodiments, the reagents include a reverse primer that is complementary to a portion of mRNA transcripts. In various embodiments, the reverse primer is a universal primer, such as any one of an oligo dT primer, oligo dU primer, or an oligo rU primer. For example, the universal primer region can be an oligo dT sequence that hybridizes with the poly A tail of messenger RNA transcripts. Therefore, the reverse primer hybridizes with a portion of mRNA transcripts and enables generation of cDNA strands through reverse transcription of the mRNA transcripts.

In various embodiments, for whole transcriptome RNA sequencing, the primers of the reaction mixture (e.g., reaction mixture 140 in FIG. 1B) include constant forward primers and constant reverse primers. The constant forward primers hybridize with the random forward primer that enabled priming off the cDNA. The constant reverse primers hybridize with a sequence of the reverse constant region, such as a PCR handle, that previously enabled reverse transcription of the mRNA transcript.

In various embodiments, primers included in the reagents (e.g., reagents 120 in FIG. 1B) or the reaction mixture (e.g., reaction mixture 140 in FIG. 1B) include additional sequences. Such additional sequences may have functional purposes. For example, a primer may include a read sequence for sequencing purposes. As another example, a primer may include a constant region. Generally, the constant region of a primer can hybridize with a complementary constant region on another nucleic acid sequence for incorporation of the nucleic acid sequence during nucleic acid amplification. For example, the constant region of a primer can be complementary to a complementary constant region of a barcode sequence. Thus, during nucleic acid amplification, the barcode sequence is incorporated into generated amplicons.

In various embodiments, instead of the primers being included in the reaction mixture (e.g., reaction mixture 140 in FIG. 1B) such primers can be included or linked to a barcode (e.g., barcode 145 in FIG. 1B). In particular embodiments, the primers are linked to an end of the barcode and therefore, are available to hybridize with target sequences of nucleic acids in the cell lysate.

In various embodiments, primers of the reaction mixture, primers of the reagents, or primers of barcodes may be added to an emulsion in one step, or in more than one step. For instance, the primers may be added in two or more steps, three or more steps, four or more steps, or five or more steps. Regardless of whether the primers are added in one step or in more than one step, they may be added after the addition of a lysing agent, prior to the addition of a lysing agent, or concomitantly with the addition of a lysing agent. When added before or after the addition of a lysing agent, the primers of the reaction mixture may be added in a separate step from the addition of a lysing agent (e.g., as exemplified in the two step workflow process shown in FIG. 1B).

A primer set for the amplification of a target nucleic acid typically includes a forward primer and a reverse primer that are complementary to a target nucleic acid or the complement thereof. In some embodiments, amplification can be performed using multiple target-specific primer pairs in a single amplification reaction, wherein each primer pair includes a forward target-specific primer and a reverse target-specific primer, where each includes at least one sequence that substantially complementary or substantially identical to a corresponding target sequence in the sample, and each primer pair having a different corresponding target sequence. Accordingly, certain methods herein are used to detect or identify multiple target sequences from a single cell sample.

Digestible Primers

Embodiments disclosed herein involve the use of digestible primers. Generally, digestible primers refer to primers that participate in a first reaction, but can be digested to prevent them from participating in a second reaction. For example, digestible primers can be primers that participate in the reverse transcription of RNA transcripts to generate cDNA, but are digested such that the digestible primers do not participate in subsequent reactions involving the cDNA (e.g., amplification of cDNA). In various embodiments, the step of digestion reduces or eliminates the presence of digestible primers (e.g., digestible primers that are primed on RNA transcripts, digestible primers that have formed undesired byproducts, and/or digestible primers that have misprimed genomic DNA). In some embodiments, digestible primers are reverse primers. In some embodiments, digestible primers are gene specific primers.

In particular embodiments, digestible primers have one of the following characteristics: A) one or more ribonucleotide nucleobases, B) one or more uracil nucleobases, C) a repeating deoxyuridine sequence (e.g., oligo dUracil or oligo dU), or D) a repeating ribouridine sequence (e.g., oligo rUracil or oligo rU).

In various embodiments, digestible primers include one or more ribonucleotide nucleobases, hereafter referred to as a “ribonucleotide primer.” In various embodiments, every nucleobase of a ribonucleotide primer are ribonucleotide nucleobases. In various embodiments, a ribonucleotide primer includes a combination of deoxyribonucleotide and ribonucleotide nucleobases. In various embodiments, ribonucleotide primers have more ribonucleotide nucleobases than deoxyribonucleotide nucleobases. In various embodiments, at least 60% of nucleobases of a ribonucleotide primer are ribonucleotide nucleobases. In various embodiments, at least 70% of nucleobases of a ribonucleotide primer are ribonucleotide nucleobases. In various embodiments, at least 80% of nucleobases of a ribonucleotide primer are ribonucleotide nucleobases. In various embodiments, at least 90% of nucleobases of a ribonucleotide primer are ribonucleotide nucleobases. In various embodiments, between 55 and 90% of nucleobases of a ribonucleotide primer are ribonucleotide nucleobases. In various embodiments, between 60 and 85% of nucleobases of a ribonucleotide primer are ribonucleotide nucleobases. In various embodiments, between 70 and 80% of nucleobases of a ribonucleotide primer are ribonucleotide nucleobases.

In various embodiments, ribonucleotide primers have more deoxyribonucleotide nucleobases than ribonucleotide nucleobases. In various embodiments, at least 60% of nucleobases of a ribonucleotide primer are deoxyribonucleotide nucleobases. In various embodiments, at least 70% of nucleobases of a ribonucleotide primer are deoxyribonucleotide nucleobases. In various embodiments, at least 80% of nucleobases of a ribonucleotide primer are deoxyribonucleotide nucleobases. In various embodiments, at least 90% of nucleobases of a ribonucleotide primer are deoxyribonucleotide nucleobases. In various embodiments, between 55 and 90% of nucleobases of a ribonucleotide primer are deoxyribonucleotide nucleobases. In various embodiments, between 60 and 85% of nucleobases of a ribonucleotide primer are deoxyribonucleotide nucleobases. In various embodiments, between 70 and 80% of nucleobases of a ribonucleotide primer are deoxyribonucleotide nucleobases.

In various embodiments, every other base of a ribonucleotide primer are ribonucleotide nucleobases. In various embodiments, the ribonucleotide primer comprises a ribonucleotide nucleobase every 3 nucleobases. In various embodiments, the ribonucleotide primer comprises a ribonucleotide nucleobase every 4 nucleobases. In various embodiments, the ribonucleotide primer comprises one ribonucleotide nucleobase every 5 nucleobases, every 6 nucleobases, every 7 nucleobases, every 8 nucleobases, every 9 nucleobases, or every 10 nucleobases.

In various, digestible primers have one or more uracil nucleobases, hereafter referred to as “uracil primers.” In various embodiments, uracil primers have combination of deoxyribonucleotides and ribonucleotides nucleobases. In some embodiments, one or more thymidine nucleobases of a deoxyribonucleotide primer can be replaced with uracil to generate a uracil primer. In some embodiments, all thymidine nucleobases of a deoxyribonucleotide primer can be replaced with uracils to generate a uracil primer. In various embodiments, a uracil primer has more deoxyribonucleotide nucleobases than uracil nucleobases. In some embodiments, a uracil primer has more uracil nucleobases than deoxyribonucleotide nucleobases. In various embodiments, every other base of a uracil primer is a uracil nucleobase. In various embodiments, the uracil primer comprises a uracil nucleobase every 3 nucleobases. In various embodiments, the uracil primer comprises a uracil nucleobase every 4 nucleobases. In various embodiments, the uracil primer comprises a uracil nucleobase every 5 nucleobases, every 6 nucleobases, every 7 nucleobases, every 8 nucleobases, every 9 nucleobases, or every 10 nucleobases.

In various embodiments, at least 30% of nucleobases of a uracil primer are deoxyribonucleotide nucleobases. In various embodiments, at least 40% of nucleobases of a uracil primer are deoxyribonucleotide nucleobases. In various embodiments, at least 50% of nucleobases of a uracil primer are deoxyribonucleotide nucleobases. In various embodiments, at least 60% of nucleobases of a uracil primer are deoxyribonucleotide nucleobases. In various embodiments, at least 70% of nucleobases of a uracil primer are deoxyribonucleotide nucleobases. In various embodiments, at least 80% of nucleobases of a uracil primer are deoxyribonucleotide nucleobases. In various embodiments, at least 90% of nucleobases of a uracil primer are deoxyribonucleotide nucleobases. In various embodiments, at least 95% of nucleobases of a uracil primer are deoxyribonucleotide nucleobases. In various embodiments, between 40 and 95% of nucleobases of a uracil primer are deoxyribonucleotide nucleobases. In various embodiments, between 50 and 90% of nucleobases of a uracil primer are deoxyribonucleotide nucleobases. In various embodiments, between 60 and 90% of nucleobases of a uracil primer are deoxyribonucleotide nucleobases. In various embodiments, between 60 and 80% of nucleobases of a uracil primer are deoxyribonucleotide nucleobases. In various embodiments, between 70 and 90% of nucleobases of a uracil primer are deoxyribonucleotide nucleobases. In various embodiments, between 70 and 80% of nucleobases of a uracil primer are deoxyribonucleotide nucleobases. In various embodiments, the uracil primer has a sequence comprising two or more consecutive uracil nucleobases. In various embodiments, the uracil primer has a sequence comprising three or more consecutive uracil nucleobases. In various embodiments, the uracil primer has a sequence comprising four or more, five or more, six or more, seven or more, eight or more, nine or more, or ten or more consecutive uracil nucleobases.

In various embodiments, the digestible primer having one or more uracil nucleobases is a gene specific primer. Here, the digestible primer would be designed in accordance with the target sequence on the specific gene. For example, based on the presence of an adenosine in the target sequence on the specific gene, the complementary base in the digestible uracil primer would be designed as a uracil. Thus, in such embodiments, the locations of uracil nucleobases in the uracil primer would be based on the target sequence and not positioned in any pattern.

In various, digestible primers have a repeating deoxyuridine sequence, hereafter referred to as “oligo dU primers.” In various embodiments, the repeating deoxyuridine sequence comprises three or more consecutive deoxyuridine nucleobases. In various embodiments, the repeating deoxyuridine sequence comprises four or more consecutive deoxyuridine nucleobases. In various embodiments, the repeating deoxyuridine sequence comprises five or more consecutive deoxyuridine nucleobases. In various embodiments, the repeating deoxyuridine sequence comprises six or more consecutive deoxyuridine nucleobases. In various embodiments, the repeating deoxyuridine sequence comprises seven or more consecutive deoxyuridine nucleobases. In various embodiments, the repeating deoxyuridine sequence comprises eight or more consecutive deoxyuridine nucleobases. In various embodiments, the repeating deoxyuridine sequence comprises nine or more consecutive deoxyuridine nucleobases. In various embodiments, the repeating deoxyuridine sequence comprises ten or more consecutive deoxyuridine nucleobases. In various embodiments, the repeating deoxyuridine sequence comprises eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, twenty five or more, twenty six or more, twenty seven or more, twenty eight or more, twenty nine or more, or thirty or more consecutive deoxyuridine nucleobases. In various embodiments, the repeating deoxyuridine sequence comprises between 5 and 30 consecutive deoxyuridine nucleobases. In various embodiments, the repeating deoxyuridine sequence comprises between 8 and 25 consecutive deoxyuridine nucleobases. In various embodiments, the repeating deoxyuridine sequence comprises between 12 and 18 consecutive deoxyuridine nucleobases.

In various embodiments, an oligo dU primer comprises a V or VN sequence, where “V” is any of an adenine (A), guanine (G), or cytosine (C) nucleobase and “N” is any of adenine (A), guanine (G), cytosine (C), or thymine (T) nucleobase. In various embodiments, the oligo dU primer terminates in the V or VN sequence (e.g., 3′ end of oligo dU contains the V or VN sequence).

In various, digestible primers have a repeating ribouridine sequence, hereafter referred to as “oligo rU primers.” In various embodiments, the repeating ribouridine sequence comprises three or more consecutive ribouridine nucleobases. In various embodiments, the repeating ribouridine sequence comprises four or more consecutive ribouridine nucleobases. In various embodiments, the repeating ribouridine sequence comprises five or more consecutive ribouridine nucleobases. In various embodiments, the repeating ribouridine sequence comprises six or more consecutive ribouridine nucleobases. In various embodiments, the repeating ribouridine sequence comprises seven or more consecutive ribouridine nucleobases. In various embodiments, the repeating ribouridine sequence comprises eight or more consecutive ribouridine nucleobases. In various embodiments, the repeating ribouridine sequence comprises nine or more consecutive ribouridine nucleobases. In various embodiments, the repeating ribouridine sequence comprises ten or more consecutive ribouridine nucleobases. In various embodiments, the repeating ribouridine sequence comprises eleven or more, twelve or more, thirteen or more, fourteen or more, fifteen or more, sixteen or more, seventeen or more, eighteen or more, nineteen or more, twenty or more, twenty one or more, twenty two or more, twenty three or more, twenty four or more, twenty five or more, twenty six or more, twenty seven or more, twenty eight or more, twenty nine or more, or thirty or more consecutive ribouridine nucleobases. In various embodiments, the repeating ribouridine sequence comprises between 5 and 30 consecutive ribouridine nucleobases. In various embodiments, the repeating ribouridine sequence comprises between 8 and 25 consecutive ribouridine nucleobases. In various embodiments, the repeating ribouridine sequence comprises between 12 and 18 consecutive ribouridine nucleobases.

In various embodiments, an oligo rU primer comprises a V or VN sequence, where “V” is any of an adenine (A), guanine (G), or cytosine (C) nucleobase and “N” is any of adenine (A), guanine (G), cytosine (C), or thymine (T) nucleobase. In various embodiments, the oligo rU primer terminates in the V or VN sequence (e.g., 3′ end of oligo dU contains the V or VN sequence).

Example System and/or Computer Embodiments

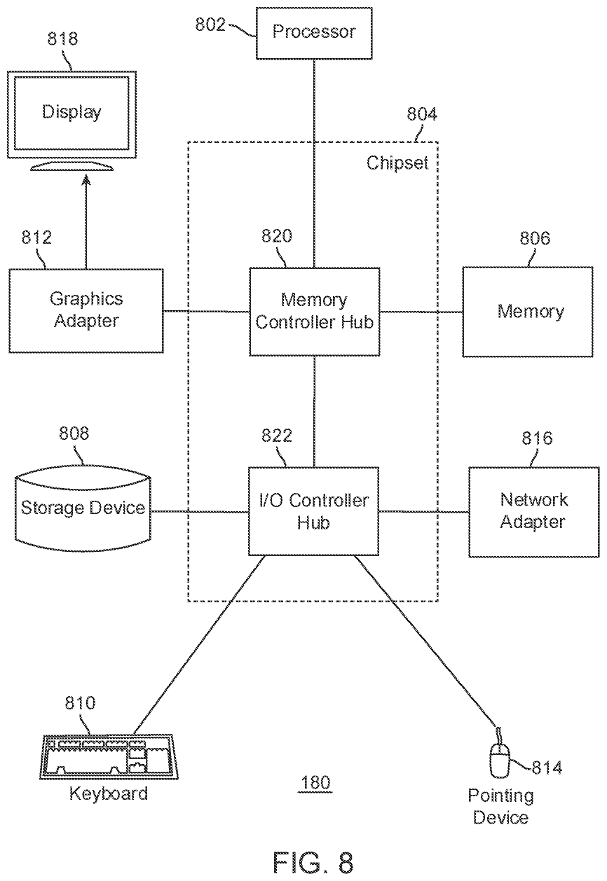

FIG. 8 depicts an example computing device (e.g., computing device 180 shown in FIG. 1A) for implementing system and methods described in reference to FIGS. 1-7. For example, the example computing device 180 is configured to perform the in silico steps of read alignment 215 and/or characterization 220. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

FIG. 8 illustrates an example computing device 180 for implementing system and methods described in FIGS. 1-7. In some embodiments, the computing device 180 includes at least one processor 802 coupled to a chipset 804. The chipset 804 includes a memory controller hub 820 and an input/output (I/O) controller hub 822. A memory 806 and a graphics adapter 812 are coupled to the memory controller hub 820, and a display 818 is coupled to the graphics adapter 812. A storage device 808, an input interface 814, and network adapter 816 are coupled to the I/O controller hub 822. Other embodiments of the computing device 180 have different architectures.

The storage device 808 is a non-transitory computer-readable storage medium such as a hard drive, compact disk read-only memory (CD-ROM), DVD, or a solid-state memory device. The memory 806 holds instructions and data used by the processor 802. The input interface 814 is a touch-screen interface, a mouse, track ball, or other type of input interface, a keyboard, or some combination thereof, and is used to input data into the computing device 180. In some embodiments, the computing device 180 may be configured to receive input (e.g., commands) from the input interface 814 via gestures from the user. The graphics adapter 812 displays images and other information on the display 818. For example, the display 818 can show metrics pertaining to the generated libraries (e.g., DNA or RNA libraries) and/or any characterization of single cells. The network adapter 816 couples the computing device 180 to one or more computer networks.

The computing device 180 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 808, loaded into the memory 806, and executed by the processor 802.

The types of computing devices 180 can vary from the embodiments described herein. For example, the computing device 180 can lack some of the components described above, such as graphics adapters 812, input interface 814, and displays 818. In some embodiments, a computing device 180 can include a processor 802 for executing instructions stored on a memory 806.

The methods of aligning sequence reads and characterizing libraries and/or cells can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of displaying any of the datasets and execution and results of this invention. Such data can be used for a variety of purposes, such as patient monitoring, treatment considerations, and the like. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

Example Kit Embodiments

Also provided herein are kits for performing single cell analysis of RNA transcripts and genomic DNA of individual or populations of cells. The kits may include one or more of the following: fluids for forming emulsions (e.g., carrier phase, aqueous phase), barcoded beads, micro fluidic devices for processing single cells, reagents for lysing cells and releasing cell analytes, reaction mixtures for performing nucleic acid amplification reactions, and instructions for using any of the kit components according to the methods described herein. In particular embodiments, the kits include digestible primers that can be used for performing reverse transcription of RNA transcripts as well as agents for digesting the digestible primers to prevent the involvement of the digestible primers in subsequent reactions, such as nucleic acid amplification reactions.

### Additional Embodiments

Disclosed herein are methods, systems, and apparati involving primers containing a mix of deoxyribonucleotide bases and ribonucleotide bases. Disclosed herein is a novel primer design to remove reverse transcription primers. Primers are used to synthesize cDNA from an RNA template in reverse transcription; however, unless these same primers are removed for future reactions such as a PCR reaction, they can participate in the amplification. For the single cell approach on Tapestri®, reverse transcription is performed in the first droplet followed by merging droplets to introduce reagents for barcoding PCR.

PCR is performed in this merged droplet so the entirety of cellular components and reagents from the first droplet are present in the second droplet. A method to remove the reverse transcription primer results in less crosstalk between DNA and RNA, and less primer byproducts, and more accurate gene expression. Primers that contain bases such as ribonucleotides or uracils, can be cleaved at those base sites to remove them from future reactions without lowering the priming specificity. By adding ribonucleotide bases every 3-4 bases in the primer, RNaseH can be used to remove the primer from future reactions without lowering the priming specificity.

In creating cDNA from mRNA in the presence of gDNA, an oligo dT primer used for reverse transcription primes the gDNA. The presence of RNaseH with an RT primer containing rU bases amongst the dT bases stops the primer from extending when hybridized to a DNA template. Also, after creating the cDNA strand, the primer is unaltered while duplexed to the RNA template.

However, if a second strand is made, the reverse transcription primer duplexes with DNA allowing for RNaseH to cleave at the ribonucleic bases. Additionally, the primer does not participate in future PCR reactions which could introduce PCR bias to the transcript count. This also can be achieved by using uracils in the oligo dT primer and including UDG in the PCR reactions.

In the case of targeted priming for cDNA synthesis, adding ribonucleic bases in the gene specific primer stops the gene specific primer from performing in any reaction other than reverse transcription. It extends cDNA but when the gene specific primer primes to DNA, the RNaseH cleaves it. Adding these ribonucleic bases only in the reverse gene specific primer, allows them only work for first stand synthesis of RNA but not the PCR resulting in more accurate gene counts. The tail sequence can only contain deoxyribonucleotide bases so it acts as the reverse primer for a more unbiased exponential amplification. Also, similarly to the mRNA approach, the gene specific primers designed for transcripts are not able to extend gDNA without being cleaved so no product is amplifiable.

An issue with large plexy gene specific panels is the primer byproducts that are created during these reactions, both reverse transcription and PCR. With primers that are cleaved once they are read through the first time, they do not form primer dimers from these reverse primers.

Another advantage to these primers is the ability to use molecular tags where the molecule is tagged during the reverse transcription but not during PCR. As a result, only RNA amplicons have a tag and there is only one tag per cDNA molecule synthesized.

While the instant disclosure provides a specific example, it is understood by one of ordinary skill in the art that the disclosed principles are not limited thereto and may be implemented independently of the Tapstri™, Miseg™ and Novaseg™ devices.

### EXAMPLES

### Example 1: RNA Base Primers for Targeted Sequencing

RNA and DNA libraries were generated from single cell analysis using either 1) DNA base primers or 2) ribonucleotide primers. Single cells were processed using the workflow described in FIG. 1B (e.g., Tapestri® workflow). The primers (e.g., solely deoxyribonucleotide primers or ribonucleotide primers) were added as the reagents during the cell encapsulation step. RNase H was further added as a part of the reaction mixture such that ribonucleotide primers added during the encapsulation step are digested. PCR cycles were subsequently performed to amplify the amplicons. Ribonucleotide base primers were designed for a 50 plex reaction whereas deoxyribonucleotide primers were designed for a 88 plex reaction. Generally, Example 1 describes the targeted sequencing schematic shown in FIGS. 4A-4B.

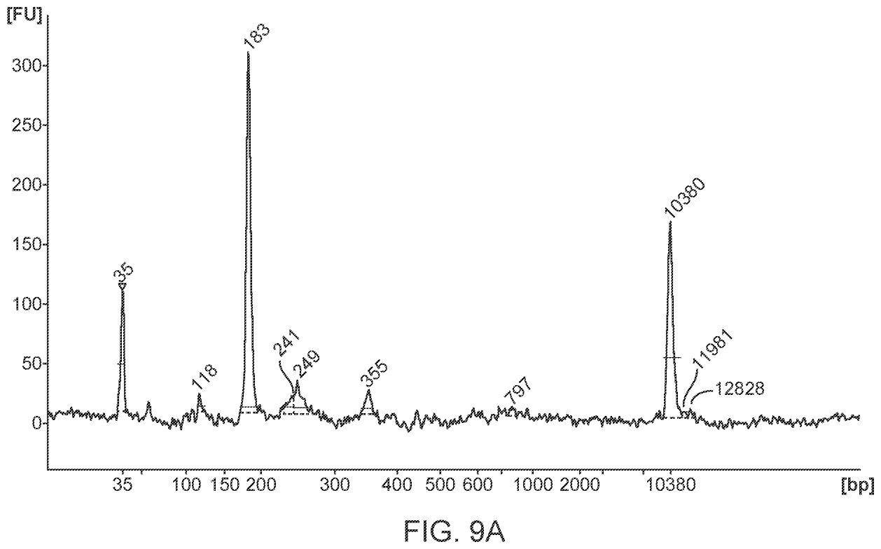

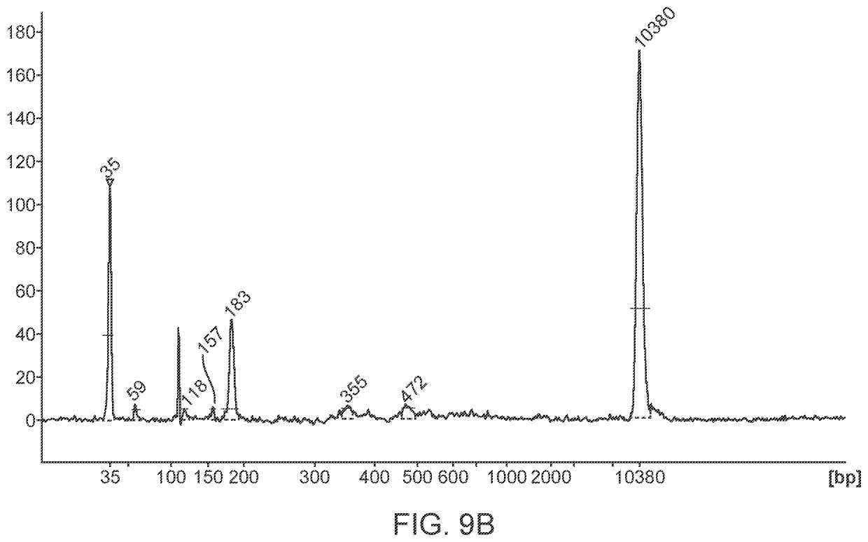

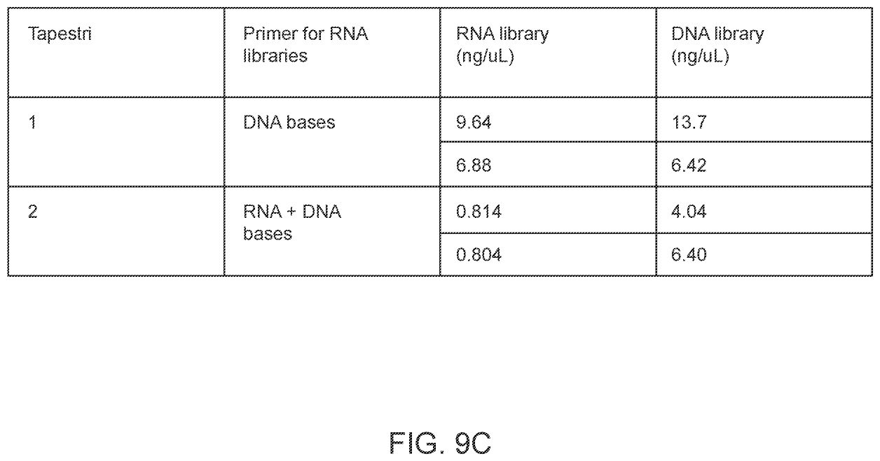

FIG. 9A depicts generated products as a result of implementation of DNA base primers for targeted RNA sequencing. FIG. 9B depicts generated products as a result of implementation of ribonucleotide primers for targeted RNA sequencing. Generally, in comparing FIGS. 9A and 9B, less primer byproduct was observed in RNA libraries using digestible ribonucleotide primers that were digested using RNaseH. Specifically, primer byproduct is observed at ˜230-250 base pairs. Here, FIG. 9B (digestible ribonucleotide primers) shows limited to no presence of primer byproducts whereas FIG. 9A (deoxyribonucleotide primers) shows presence of primer byproducts, indicating that the implementation of ribonucleotide primers that are subsequently digested using RNaseH reduces presence of primer byproducts.

Furthermore, the desired product is observed between 400-500 base pairs. FIG. 9B (ribonucleotide primers) shows presence of desired product (e.g., at ˜472 base pairs) whereas FIG. 9A (deoxyribonucleotide primers) shows a lack of presence of the desired product, indicating that the implementation of ribonucleotide primers that are subsequently digested using RNaseH increases the presence of desired product. DNA libraries (88 plex) were not affected by the use of ribonucleotide primers used for RNA libraries (not shown).

FIG. 9C depicts quantitative amounts of generated products as a result of implementation of deoxyribonucleotide or ribonucleotide primers for targeted sequencing. Here, DNA library yields were generally not affected by the use of deoxyribonucleotide or ribonucleotide base primers. RNA libraries using ribonucleotide primers demonstrated lower yield; however, less primer byproduct was observed in the bioanalyzer trace, which contributes towards the lower yields. If needed, additional PCR cycles can be performed to further increase the yield of RNA libraries that are generated using ribonucleotide primers.

### Example 2: Uracil Priming for Whole Transcriptome Sequencing

RNA and DNA libraries were generated from single cell analysis using either 1) oligo dT primers or 2) oligo dU primers. Single cells were processed using the workflow described in FIG. 1B (e.g., Tapestri® workflow). Table 1 below documents the reagents included when encapsulating single cells. The cDNA synthesis was performed with oligo dT or oligo dU. Table 2 below documents the agents included in the reaction mixture for cell barcoding and target amplification. Notably, the cDNA product was amplified with ABCB7 primers on the qPCR instrument using a binding dye. Generally, Example 2 describes the whole transcriptome schematic shown in FIGS. 7A-7B.

TABLE 1
Reagent mixture

| Volume (μL) | Reagent |
|---|---|
| 1 | Maxima |
| 0.5 | Bsu |
| 4 | 5X Maxima buffer |
| 1 | dNTPs (10 mM, Thermo) |
| 1 | DTT (100 mM, Thermo) |
| 1 | Ribonuclease inhibitor (Thermo) |
| 0.1 | RNaseH (Thermo) |
| 1 | UHR (100 ng/uL) |
| 1.5 | Fwd A RP6r (random hexamer with |
| | RNA base at the 3′ end) (25 uM) |
| 1 | Oligo dT or oligo dU (50 uM) |
| Up to 20 uL | dH₂O |

Temperature ramping of 1) 50° C. for 15 minutes, 2) 25° C. for 10 minutes, 3) 50° C. for 35 minutes, and 4) 85° C. for 10 minutes.

TABLE 2
Reaction mixture

| Volume (μL) | Reagent |
|---|---|
| 1 | Evagreen |
| 0.4 | ROX |
| 4 | RT rxn |
| 10 | Library mix |
| 1.6 | ABCB7 primers (forward + |
| | reverse at 2.5 uM each) |

PCR involved 40 cycles of the following protocol: 1) 95° C. for 3 minutes, 2) 98° C. for 20 seconds, 3) 62° C. for 20 seconds, 4) 72° C. for 45 seconds, and 5) 72° C. for 2 minutes.

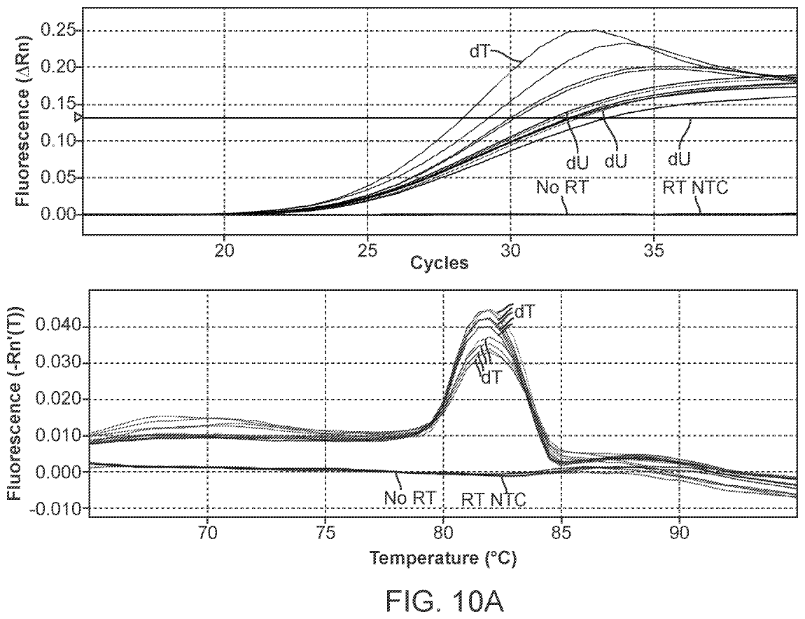

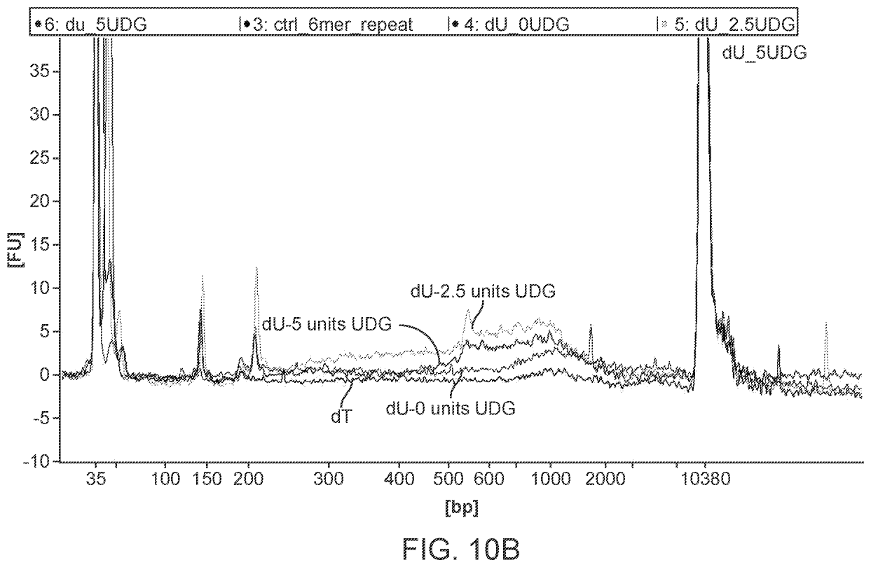

FIG. 10A depicts qPCR and melting temperature plots identifying generated products as a result of implementation of uracil primers for whole transcriptome sequencing. Although amplification was not as good as oligo dT (top panel of FIG. 10A), the melt curve (bottom panel FIG. 10A) shows that it resulted in the same product which was not observed in the no RT reaction or the no template control (NTC) reaction.

FIG. 10B depicts generated products as a result of implementing various concentrations of uracil-DNA glycosylase (UDG) enzyme. Here, 12 uL of the cDNA was used for bulk library preparation with a cell barcode for 18 cycles. 0 units, 2.5 units, or 5 units of thermostable UDG were included in the library preparation. Notably, desired product is observed between 300 bp to 2000 bp. Thus, libraries were observed with oligo dU used for cDNA synthesis

Libraries were pooled equivolume and sequenced. Metrics shown below in Table 3 and Table 4 demonstrate that reads were generated from RNA. Notably, as shown in Table 3, use of oligo dU and various concentrations of UDG (e.g., 2.5 units or 5 units of UDG) resulted in significant library yield (e.g., 0.4 ng/uL and 0.388 ng/uL respectively) with corresponding sequence reads. Furthermore, as shown in Table 4, use of oligo dU and 5 units UDG for digesting the oligo dU resulted in higher % reads after trimming, % of oligo dT/dU reads, % of reads with forward primer, % mapped, and % reads with valid cell barcode in comparison to the control group (e.g., use of oligo dT).

TABLE 3
Library yield and sequencing reads for
oligo dU primers and UDG digestion

| Sample | Library yield (ng/uL) | Sequencing Reads |
|---|---|---|
| dT | 0.276 | 9290 |
| dU - 0 units UDG | 0.276 | 38110 |
| dU - 2.5 units UDG | 0.400 | 78046 |
| dU - 5 units UDG | 0.388 | 48507 |
| No RT | Too low | 650 |
| Library PCR NTC | 0.184 | 70 |

TABLE 4
Metrics of sequence reads as a result of implementing
oligo dU primers and UDG digestion.

| | % reads | % oligo | Reads with | | % reads |
|---|---|---|---|---|---|
| | after | dT/dU | forward | % mapped | with valid |
| Sample | trimming | reads | primer (%) | reads | cell barcode |
| dT | 68.72% | 26.65% | 26.65% | 2.22% | 2.19% |
| dU - 5 | 80.12% | 73.20% | 73.20% | 10.69% | 10.58% |
| units UDG | | | | | |

### Example 3: Uracil and RNA Base Priming for Whole Transcriptome Sequencing

RNA and DNA libraries were generated from single cell analysis using either 1) oligo dT primers, 2) oligo dU primers, or 3) oligo rU primers. Single cells were processed using the workflow described in FIG. 1B (e.g., Tapestri® workflow). Table 5 below documents the reagents included when encapsulating single cells. Generally, Example 3 describes the whole transcriptome schematic shown in FIGS. 6A-6B (oligo rU) and FIGS. 7A-7B (oligo dU).

TABLE 5
Reagent mixture for whole transcriptome sequencing

| Volume (μL) | Reagent |
|---|---|
| 1 | SSIV |
| 0.5 | Bsu |
| 4 | 5X buffer |
| 1 | dNTPs (10 mM, Thermo) |
| 1 | DTT (100 mM, Thermo) |
| 1 | Ribonuclease inhibitor (Thermo) |
| 0.1 | RNaseH (Thermo) |
| 1 | UHR (100 ng/uL) |
| 1.5 | Fwd A RP6r (25 uM) |
| 0.2 | Oligo dT (250 uM) or dU or rU |
| Up to 20 uL | dH₂O |

Temperature ramping of 1) 50° C. for 15 minutes, 2) 25° C. for 10 minutes, 3) 50° C. for 35 minutes, and 4) 85° C. for 10 minutes.

Linear amplification was performed with bulk bead oligo using a barcode mix with 51 C as outer barcode annealing temp and 2 uL RT product input. Library amplification was performed with 15 uL input for 18 cycles.

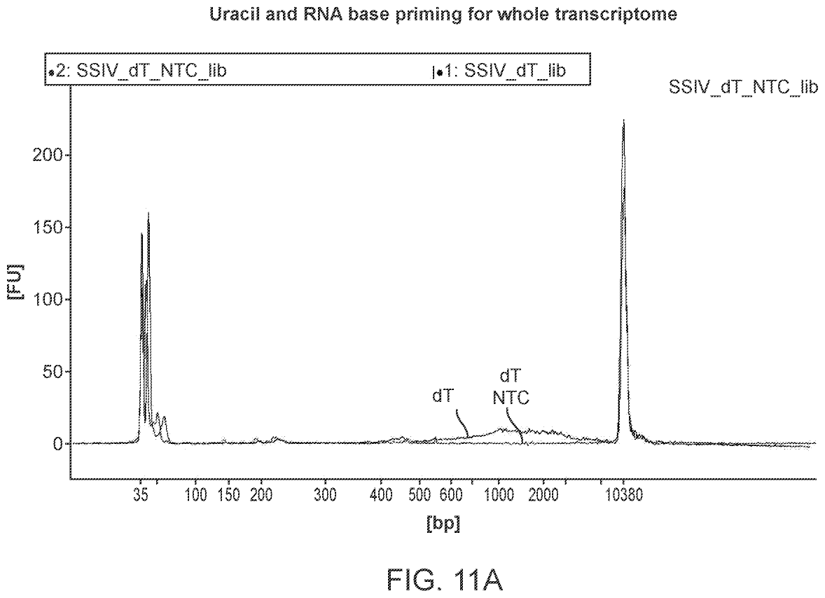

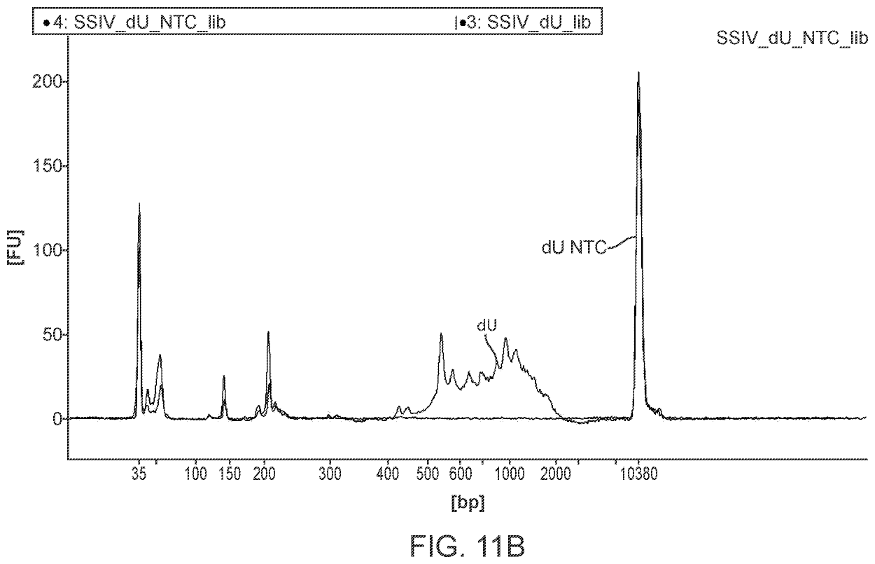

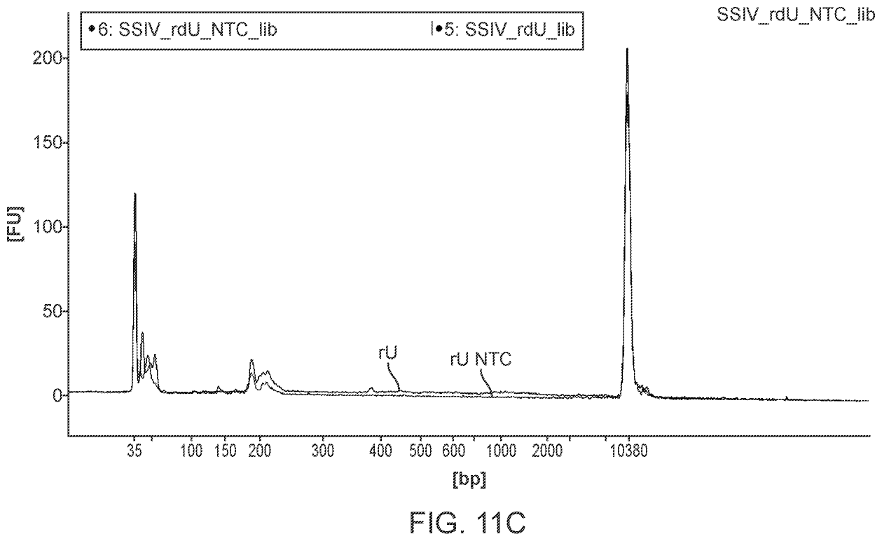

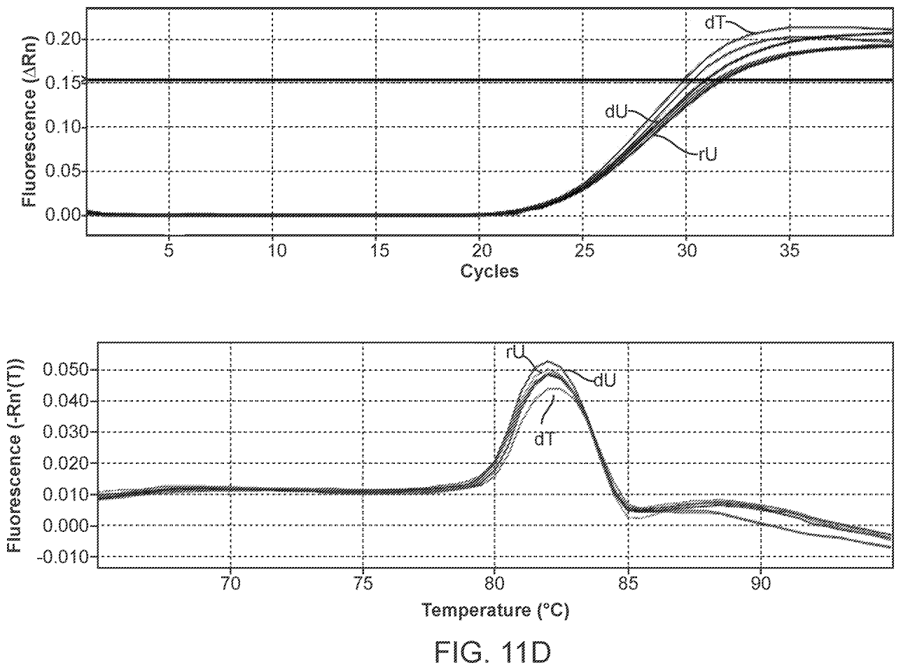

FIGS. 11A-11C depict generated products as a result of implementing oligo dT, oligo dU, or oligo rU primers. Specifically, FIG. 11A depicts products generated when using oligo dT or a no template control, FIG. 11B depicts products generated when using oligo dU or a no template control, and FIG. 11C depicts products generated when using oligo rU primers (“rU” as referenced in FIG. 11C) or a no template control. Generally, libraries were observed with oligo dT, dU, and rU used for cDNA synthesis. Notably, desired product is observed between 300 bp to 2000 bp, especially as can be observed in FIG. 11B.

Table 6 below summarizes the barcode and library yield for each of the different groups. In particular, use of each of oligo dT, oligo dU, or oligo rU base primers resulted in barcode yield whereas their corresponding no template controls (NTCs) resulted in non-detectable barcode yield. Similarly, use of each of oligo dT, oligo dU, or oligo rU base primers resulted in higher library yield in comparison to their corresponding no template controls (NTCs).

TABLE 6
Barcode and library yields.

| Sample | Barcode yield (ng/uL) | Library yield (ng/uL) |
|---|---|---|
| SSIV-dT | 0.150 | 0.302 |
| SSIV-dT-NTC | Too low | 0.226 |
| SSIV-dU | 0.200 | 0.604 |
| SSIV-dU-NTC | Too low | 0.178 |
| SSIV-rU | 0.102 | 0.238 |
| SSIV-rU-NTC | Too low | 0.168 |

Another experiment was conducted that synthesized cDNA using oligo dT, oligo dU, or oligo rU. Table 7 below documents the reagents included when encapsulating single cells. The cDNA product was amplified with ABCB7 primers on the qPCR instrument using a binding dye.

TABLE 7
Reagent mixture

| Volume (μL) | Reagent |
|---|---|
| 4 | 5X buffer |
| 0.6 | 10% NP40 |
| 1 | dNTPs (10 mM, Thermo) |
| 1.5 | Betaine (5M) |
| 0.25 | Maxima H minus RT |
| 1 | revA-dT18bV (10 uM) |
| 0.1 | Rnase H |
| 1 | 100 ng/uL UHR |
| 10.55 | dH₂O |

FIG. 11D depicts qPCR and melting temperature plots identifying generated products as a result of implementing oligo dT, oligo dU, or oligo rU primers for whole transcriptome sequencing. Amplification appeared similar between oligo dT, oligo dU, and oligo rU primers with these RT conditions. Additionally, the melting temperature plots demonstrate similar product formation across the oligo dT, oligo dU, and oligo rU primers.

### Example 4: Nested Uracil and RNA Base Priming

Libraries are generated from single cell analysis using either 1) oligo dT primers, 2) oligo dU primers, or 3) oligo rU primers. Single cells were processed using the workflow described in FIG. 1B (e.g., Tapestri® workflow). Table 8 below documents the reagents for including when encapsulating single cells. Table 9 below documents the agents for including in the reaction mixture for cell barcoding and target amplification. Generally, Example 4 describes the nested targeted sequencing schematic shown in FIGS. 5A-5B.

TABLE 8
Reagent mixture for nested targeted sequencing

| Volume (μL) | Reagent |
|---|---|
| 5 | SSIV |
| 20 | 5X buffer |
| 5 | dNTPs (10 mM, Thermo) |
| 5 | DTT (100 mM, Thermo) |
| 5 | RNase inhibitor |
| 10 | 10% NP40 |
| 7.3 | 200 uM GSP outer primer for RNA |
| | library (includes either RNA bases |
| | or uracils) |
| 0.2165 | 20 mg/mL proteinase K |
| 42.4835 | dH₂O |

Preheat thermocycler: 1) 50° C. for 60 minutes and 2) 80° C. for 10 minutes.

TABLE 9
Reaction mixture

| Volume (μL) | | Reagent |
|---|---|---|
| 0.5-20 | units | Thermostable RNaseH or UDG |
| 3.125 | | 200 uM GSP rev RNA (inner |
| | | primer for RNA library) |
| 2.5 | | 25 uM GSP fwd DNA |
| 0.625 | | 200 uM GSP fwd RNA |
| 3.125 | uL | 200 uM GSP rev DNA |
| Up to 300 | uL | Barcoding MM v2 |

PCR nucleic acid amplification involves: 1) 1 cycle of 98° C. for 30 seconds, 2) 20 cycles of 98° C. for 10 seconds, 3) 72° C. for 45 seconds, 4) 20 cycles of 98° C. for 30 seconds, 5) 61° C. for 30 seconds, 6) 72° C. for 45 seconds, 7) 72° C. for 3 minutes, and 8) hold at 4° C.

Generally, digesting the primer used for RT results in less primer byproduct, increase the specificity of on target reads, and improve the gene count accuracy.

What is claimed is:

1. A method for generating a nucleic acid library, the method comprising:
obtaining RNA and DNA from a single cell within a droplet;
priming the RNA from the single cell using a digestible primer within the droplet;
generating cDNA comprising the digestible primer from the primed RNA within the droplet;
digesting the digestible primer; and
sequencing at least the cDNA and the DNA of the single cell or sequences derived from the cDNA and the DNA of the single cell.

2. The method of claim 1, wherein the digestible primer comprises one of:
A) one or more ribonucleotide nucleobases,
B) one or more uracil nucleobases,
C) a repeating deoxyuridine sequence, or
D) a repeating ribouridine sequence,
wherein digesting the digestible primer occurs subsequent to generating the cDNA and prior to a second cycle of nucleic acid amplification,
wherein digesting the digestible primer comprises exposing the digestible primer to a RNase or uracil-DNA glycosylase.

3. The method of claim 1, wherein the digestible primer comprises one or more ribonucleotide nucleobases.

4. The method of claim 3, wherein the digestible primer comprises a combination of deoxyribonucleotide and ribonucleotide nucleobases.

5. The method of claim 1 or 3, wherein the digestible primer comprises a ribonucleotide nucleobase every 2 nucleobases.

6. The method of claim 1 or 3, wherein the digestible primer comprises a ribonucleotide nucleobase every 3 nucleobases.

7. The method of claim 1 or 3, wherein the digestible primer comprises a ribonucleotide nucleobase every 4 nucleobases.

8. The method of claim 1 or 3, wherein the digestible primer comprises a ribonucleotide nucleobase every 5 nucleobases, every 6 nucleobases, every 7 nucleobases, every 8 nucleobases, every 9 nucleobases, or every 10 nucleobases.

9. The method of claim 1, wherein the digestible primer comprises at least 3 consecutive ribouridine nucleobases.

10. The method of claim 1, wherein the digestible primer comprises between 5 and 30 consecutive ribouridine nucleobases.

11. The method of any one of claims 1 and 3-9, wherein digesting the digestible primer comprises exposing the digestible primer to a RNase.

12. The method of claim 11, wherein the RNase is one of RNase A or RNase H.

13. The method of claim 1, wherein the digestible primer comprises one or more uracil nucleobases.

14. The method of claim 1 or 13, wherein the digestible primer comprises a uracil nucleobase every 3 nucleobases.

15. The method of claim 1 or 13, wherein the digestible primer comprises a uracil nucleobase every 4 nucleobases.

16. The method of claim 1 or 13, wherein the digestible primer comprises a uracil nucleobase every 5 nucleobases, every 6 nucleobases, every 7 nucleobases, every 8 nucleobases, every 9 nucleobases, or every 10 nucleobases.

17. The method of claim 1, wherein the digestible primer comprises at least 3 consecutive deoxyuridine nucleobases.

18. The method of claim 1 or 17, wherein the digestible primer comprises between 5 and 30 consecutive deoxyuridine nucleobases.

19. The method of any one of claim 1 or 13-18, wherein digesting the digestible primer comprises exposing the digestible primer to uracil-DNA glycosylase (UDG).

20. The method of any one of claims 1 and 3-19, wherein generating cDNA comprising the digestible primer from the primed RNA comprises reverse transcribing the primed RNA.

21. The method of any one of claims 1 and 3-20, wherein digesting the digestible primer occurs within a second droplet.

22. The method of any one of claims 1 and 3-21, wherein digesting the digestible primer occurs subsequent to a first cycle of nucleic acid amplification.

23. The method of any one of claims 1 and 3-22, wherein subsequent to generating cDNA and prior to digesting the digestible primer:
synthesizing a nucleic acid product derived from the cDNA, the nucleic acid product further comprising a sequence derived from a sequence of the digestible primer.

24. The method of any one of claims 1 and 3-23, wherein digesting the digestible primer occurs prior to a first cycle of nucleic acid amplification.

25. The method of claim 24, wherein subsequent to digesting the digestible primer:
synthesizing a nucleic acid product derived from the cDNA, the nucleic acid product lacking a sequence derived from a sequence of the digestible primer; and
priming the synthesized nucleic acid using a second primer different from the digestible primer.

26. The method of claim 25, wherein the second primer is a gene specific primer.

27. The method of claim 26, wherein the sequencing is a targeted sequencing.

28. The method of claim 24, wherein prior to digesting the digestible primer:
priming the cDNA using a random primer; and
synthesizing a nucleic acid product derived from the cDNA, the nucleic acid product further comprising a sequence derived from a sequence of the digestible primer.

29. The method of claim 28, wherein digesting the digestible primer occurs within the droplet.

30. The method of claim 28, wherein digesting the digestible primer occurs within a second droplet.

31. The method of any one of claims 28-30, wherein the sequencing is a whole transcriptome sequencing.

32. The method of any one of claims 1 and 3-31, further comprising: subsequent to digesting the digestible primer, performing nucleic acid amplification to generate cDNA and gDNA amplicons.

33. The method of claim 32, wherein performing nucleic acid amplification comprises incorporating cellular barcodes that indicate the single cell of origin, thereby generating cDNA amplicons comprising the cellular barcodes.

34. The method of any one of claims 1-33, wherein obtaining RNA from a single cell within a droplet comprises:
encapsulating the single cell in the droplet comprising reagents;
lysing the single cell within the droplet; and
exposing the lysed cell to conditions sufficient to release DNA from packaged chromatin.

35. The method of claim 34, wherein the reagents comprise proteinase K, and wherein exposing the lysed cell comprising exposing the lysed cell to proteinase K to release DNA from packaged chromatin.

36. The method any one of claims 1-35, wherein sequencing at least the cDNA of the single cell results in at least a 2-fold, at least a 3-fold, at least a 4-fold, or at least a 5-fold increase in percentage of mapped reads in comparison to a workflow process that implements oligo dT primers as opposed to digestible primers.

37. The method any one of claims 1-35, wherein sequencing at least the cDNA of the single cell results in at least a 2-fold, at least a 3-fold, at least a 4-fold, or at least a 5-fold increase in percentage of reads with a valid barcode in comparison to a workflow process that implements oligo dT primers as opposed to digestible primers.

38. A system for generating a nucleic acid library, the system comprising:
a device configured to perform steps comprising:
obtaining RNA and DNA from a single cell within a droplet;
priming the RNA from the single cell using a digestible primer within the droplet;
generating cDNA comprising the digestible primer from the primed RNA within the droplet;
digesting the digestible primer; and
sequencing at least the cDNA and the DNA of the single cell or sequences derived from the cDNA and the DNA of the single cell.

39. The system of claim 38, wherein the digestible primer comprises one of:
A) one or more ribonucleotide nucleobases,
B) one or more uracil nucleobases,
C) a repeating deoxyuridine sequence, or
D) a repeating ribouridine sequence,
wherein digesting the digestible primer occurs subsequent to generating the cDNA and prior to a second cycle of nucleic acid amplification,
wherein digesting the digestible primer comprises exposing the digestible primer to a RNase or uracil-DNA glycosylase.

40. The system of claim 38, wherein the digestible primer comprises one or more ribonucleotide nucleobases.

41. The system of claim 40, wherein the digestible primer comprises a combination of ribonucleotides and deoxyribonucleotides.

42. The system of claim 38 or 40, wherein the digestible primer comprises a ribonucleotide nucleobase every 2 nucleobases.

43. The system of claim 38 or 40, wherein the digestible primer comprises a ribonucleotide nucleobase every 3 nucleobases.

44. The system of claim 38 or 40, wherein the digestible primer comprises a ribonucleotide nucleobase every 4 nucleobases.

45. The system of claim 38 or 40, wherein the digestible primer comprises a ribonucleotide nucleobase every 5 nucleobases, every 6 nucleobases, every 7 nucleobases, every 8 nucleobases, every 9 nucleobases, or every 10 nucleobases.

46. The system of claim 38, wherein the digestible primer comprises at least 3 consecutive ribouridine nucleobases.

47. The system of claim 38, wherein the digestible primer comprises between 5 and 30 consecutive ribouridine nucleobases.

48. The system of any one of claims 38 and 40-47, wherein digesting the digestible primer comprises exposing the digestible primer to a RNase.

49. The system of claim 48, wherein the RNase is one of RNase A or RNase H.

50. The system of claim 38, wherein the digestible primer comprises one or more uracil nucleobases.

51. The system of claim 38 or 50, wherein the digestible primer comprises a uracil nucleobase every 3 nucleobases.

52. The system of claim 38 or 50, wherein the digestible primer comprises a uracil nucleobase every 4 nucleobases.

53. The system of claim 38 or 50, wherein the digestible primer comprises a uracil nucleobase every 5 nucleobases, every 6 nucleobases, every 7 nucleobases, every 8 nucleobases, every 9 nucleobases, or every 10 nucleobases.

54. The system of claim 38, wherein the digestible primer comprises at least 3 consecutive deoxyuridine nucleobases.

55. The system of claim 38 or 54, wherein the digestible primer comprises between 5 and 30 consecutive deoxyuridine nucleobases.

56. The system of any one of claim 38 or 50-55, wherein digesting the digestible primer comprises exposing the digestible primer to uracil-DNA glycosylase.

57. The system of any one of claims 38 and 40-56, wherein generating cDNA comprising the digestible primer from the primed RNA comprises reverse transcribing the primed RNA.

58. The system of any one of claims 38 and 40-57, wherein digesting the digestible primer occurs within a second droplet.

59. The system of any one of claims 38 and 40-58, wherein digesting the digestible primer occurs subsequent to a first cycle of nucleic acid amplification.

60. The system of any one of claims 38 and 40-59, wherein subsequent to generating cDNA and prior to digesting the digestible primer, the device is configured to perform steps comprising:
synthesizing a nucleic acid product derived from the cDNA, the nucleic acid product further comprising a sequence derived from a sequence of the digestible primer.

61. The system of any one of claims 38 and 40-60, wherein digesting the digestible primer occurs prior to a first cycle of nucleic acid amplification.

62. The system of claim 61, wherein subsequent to digesting the digestible primer, the device is configured to perform steps comprising:
synthesizing a nucleic acid product derived from the cDNA, the nucleic acid product lacking a sequence derived from a sequence of the digestible primer; and
priming the synthesized nucleic acid using a second primer different from the digestible primer.

63. The system of claim 62, wherein the second primer is a gene specific primer.

64. The system of claim 63, wherein the sequencing is a targeted sequencing.

65. The system of claim 61, wherein prior to digesting the digestible primer:
priming the cDNA using a random primer; and
synthesizing a nucleic acid product derived from the cDNA, the nucleic acid product further comprising a sequence derived from a sequence of the digestible primer.

66. The system of claim 65, wherein digesting the digestible primer occurs within the droplet.

67. The system of claim 65, wherein digesting the digestible primer occurs within a second droplet.

68. The system of any one of claims 65-67, wherein the sequencing is a whole genome sequencing.

69. The system of any one of claims 38 and 40-68, wherein the device is further configured to perform steps comprising: subsequent to digesting the digestible primer, performing nucleic acid amplification on the cDNA to generate cDNA amplicons.

70. The system of claim 69, wherein performing nucleic acid amplification comprises incorporating cellular barcodes that indicate the single cell of origin, thereby generating cDNA amplicons comprising the cellular barcodes.

71. The system of any one of claims 38-70, wherein obtaining RNA from a single cell within a droplet comprises:
encapsulating the single cell in the droplet comprising reagents;
lysing the single cell within the droplet; and
exposing the lysed cell to conditions sufficient to release DNA from packaged chromatin.

72. The system of claim 71, wherein the reagents comprise proteinase K, and wherein exposing the lysed cell comprising exposing the lysed cell to proteinase K to release DNA from packaged chromatin.

73. The system any one of claims 38-72, wherein sequencing at least the cDNA of the single cell results in at least a 2-fold, at least a 3-fold, at least a 4-fold, or at least a 5-fold increase in percentage of mapped reads in comparison to a workflow process that implements oligo dT primers as opposed to digestible primers.

74. The system any one of claims 38-72, wherein sequencing at least the cDNA of the single cell results in at least a 2-fold, at least a 3-fold, at least a 4-fold, or at least a 5-fold increase in percentage of reads with a valid barcode in comparison to a workflow process that implements oligo dT primers as opposed to digestible primers.
